(19) United States
(12) Patent Application Publication
(10) Pub. No.: US 2024/0393188 A1
(43) Pub. Date: Nov. 28, 2024

(54) TEMPERATURE MEASUREMENT DEVICE

(71) Applicant: Murata Manufacturing Co., Ltd., Nagaokakyo-shi (JP)

(72) Inventors: Yuki SAKURADA, Nagaokakyo-shi (JP); Hiroaki IDE, Nagaokakyo-shi (JP); Hironori NOTO, Nagaokakyo-shi (JP); Tomoki OUCHI, Nagaokakyo-shi (JP); Koji TANAKA, Nagaokakyo-shi (JP); Kiyoshi KURIHARA, Nagaokakyo-shi (JP)

(21) Appl. No.: 18/792,147

(22) Filed: Aug. 1, 2024

Related U.S. Application Data
(63) Continuation of application No. PCT/JP2022/045827, filed on Dec. 13, 2022.

(30) Foreign Application Priority Data
Feb. 7, 2022 (JP) 2022-017416
May 19, 2022 (JP) 2022-082429

(51) Int. Cl. G01K 13/25; A61B 18/00; A61B 18/02; A61B 18/04; G01K 1/02

(52) U.S. Cl. CPC G01K 13/25; A61B 18/02; G01K 1/026; A61B 2018/0022; A61B 2018/00351; A61B 2018/00577; A61B 2018/00791; A61B 2018/0212; A61B 2018/0262; A61B 2018/046

(57) ABSTRACT

A temperature measurement device that includes: a tube; and a temperature sensor unit within the tube. The temperature sensor unit includes a flexible sheet and a plurality of temperature sensor elements on the flexible sheet.

### CROSS REFERENCE TO RELATED APPLICATIONS

The present application is a continuation of International application No. PCT/JP2022/045827, filed Dec. 13, 2022, which claims priority to Japanese Patent Application No. 2022-017416, filed Feb. 7, 2022, and Japanese Patent Application No. 2022-082429, filed May 19, 2022, the entire contents of each of which are incorporated herein by reference.

### TECHNICAL FIELD

The present disclosure relates to temperature measurement devices and particularly to a temperature measurement device for measuring the temperature of a tubular organ in a living body.

### BACKGROUND ART

As an example of a treatment method for atrial fibrillation, left atrial ablation that cauterizes cardiac muscle is known. In left atrial ablation, there is a possibility that heat for cauterization can be propagated to the esophagus anatomically close to the heart, and that the esophagus can receive thermal damage.

To address this, a technique for measuring the temperature inside the esophagus to prevent thermal damage to the esophagus is known. For example, Patent Document 1 discloses an esophageal mapping catheter that is placed inside the esophagus during ablation, measures the temperature inside the esophagus, and gives feedback to the user, in order to prevent thermal damage to the esophagus.
Patent Document 1: Japanese Unexamined Patent Application Publication (Translation of PCT Application) No. 2010-505592

### SUMMARY OF THE DISCLOSURE

The esophageal mapping catheter described in Patent Document 1 has thick temperature sensors. In the case in which these temperature sensors are stored at high density in a hollow tool such as a catheter, the thick configuration causes gaps in the tool and hence has a problem that storing at high density is difficult.

To address this, an object of the present disclosure is to provide a temperature measurement device including temperature sensor elements constructed to be stored at high density in a limited space in a tube.

A temperature measurement device according to an aspect of the present disclosure includes: a tube; and a temperature sensor unit within the tube, wherein the temperature sensor unit includes a flexible sheet and a plurality of temperature sensor elements on the flexible sheet.

The temperature measurement device according to the present disclosure increases the density of temperature sensor elements stored in a tube.

### BRIEF DESCRIPTION OF THE DRAWINGS

FIG. 1 is a schematic perspective view of a configuration example of a temperature measurement device according to a first embodiment.

FIG. 2 is a cross-sectional view of the temperature measurement device in FIG. 1, schematically illustrating an example of a stored state of a temperature sensor unit.

FIG. 3 is a cross-sectional view of the temperature measurement device in FIG. 1, schematically illustrating an example of a deployed state of the temperature sensor unit.

FIG. 4 is a schematic diagram illustrating the configuration example of the temperature sensor unit in the stored state.

FIG. 5 is a schematic diagram illustrating the configuration example of the temperature sensor unit in the deployed state.

FIG. 6 is a schematic cross-sectional view of the temperature sensor unit taken along line VI-VI in FIG. 4.

FIG. 7 is a schematic diagram illustrating a temperature sensor unit in a stored state in a temperature measurement device according to a second embodiment.

FIG. 8 is a schematic diagram illustrating the temperature sensor unit in a deployed state in the temperature measurement device according to the second embodiment.

FIG. 9A is a schematic diagram illustrating a temperature sensor unit in a stored state in a temperature measurement device according to a third embodiment.

FIG. 9B is a schematic perspective view of the temperature sensor unit in the stored state in the temperature measurement device according to the third embodiment.

FIG. 10A is a schematic diagram illustrating the temperature sensor unit in a deployed state in the temperature measurement device according to the third embodiment.

FIG. 10B is a schematic perspective view of the temperature sensor unit in the deployed state in the temperature measurement device according to the third embodiment.

FIG. 11 is a schematic diagram illustrating a temperature sensor unit according to a modification example of the third embodiment.

FIG. 12 is a schematic cross-sectional view of a configuration example of a temperature measurement device according to a fourth embodiment.

FIG. 13 is a schematic cross-sectional view of the configuration example of the temperature measurement device according to the fourth embodiment.

FIG. 14 is a schematic cross-sectional view of a temperature sensor unit, in a stored state, of a temperature measurement device according to a fifth embodiment.

FIG. 15 is a schematic cross-sectional view of the temperature sensor unit, in a deployed state, of the temperature measurement device according to the fifth embodiment.

FIG. 16 is a schematic cross-sectional view of a balloon, in a contracted state, of a temperature measurement device according to a modification example of the fifth embodiment.

FIG. 17 is a schematic cross-sectional view of the balloon, in an intermediate state, of the temperature measurement device according to the modification example of the fifth embodiment.

FIG. 18 is a schematic cross-sectional view of the balloon, in an expanded state, of the temperature measurement device according to the modification example of the fifth embodiment.

FIG. 19 is a schematic side view of a basket catheter, in a contracted state, of a temperature measurement device according to a sixth embodiment.

FIG. 20 is a schematic side view of the basket catheter, in an expanded state, of the temperature measurement device according to the sixth embodiment.

FIG. 21 is a schematic cross-sectional view of a temperature sensor unit, in a stored state, of the temperature measurement device according to the sixth embodiment.

FIG. 22 is a schematic cross-sectional view of the temperature sensor unit, in a deployed state, of the temperature measurement device according to the sixth embodiment.

FIG. 23 is a schematic diagram illustrating a configuration example of a temperature sensor unit and a balloon of a temperature measurement device according to a seventh embodiment.

FIG. 24 is a cross-sectional view of the temperature sensor unit and the balloon taken along line XXIV-XXIV in FIG. 23.

FIG. 25 is a schematic diagram illustrating the cross section corresponding to FIG. 24 of the temperature sensor unit and the balloon in a stored state.

FIG. 26 is a cross-sectional view of the temperature sensor unit and the balloon taken along line XXVI-XXVI in FIG. 23.

FIG. 27 is a schematic diagram illustrating the cross section corresponding to FIG. 26 of the temperature sensor unit and the balloon in the stored state.

FIG. 28 is a schematic diagram illustrating a configuration example of a temperature sensor unit and a balloon according to a comparative example of the seventh embodiment.

FIG. 29 is a cross-sectional view of the temperature sensor unit and the balloon taken along line XXIX-XXIX in FIG. 28.

FIG. 30 is a schematic diagram illustrating the cross section corresponding to FIG. 29 of the temperature sensor unit and the balloon in a stored state.

FIG. 31 is a schematic diagram illustrating a configuration example of a temperature sensor unit according to a modification example of an embodiment of the present disclosure.

FIG. 32 is a schematic cross-sectional view of a configuration example of a temperature measurement device according to another modification example of an embodiment of the present disclosure.

### DESCRIPTION OF THE PREFERRED EMBODIMENTS

### Background Leading to Present Disclosure

The esophageal mapping catheter described in Patent Document 1 has thick temperature sensors. In the case in which these temperature sensors are stored at high density in a hollow tool such as a catheter, the thick configuration causes gaps in the tool and hence has a problem that storing at high density is difficult.

The inventors had conducted research to solve the above problem and conceived a temperature measurement device that increases the density of temperature sensor elements stored in a tube.

Hereinafter, temperature measurement devices according to embodiments of the present disclosure will be described with reference to the attached drawings. In the following embodiments, the same or similar constituents are denoted by the same symbols. To facilitate understanding of explanation, the shape, dimensions, positional relationship of each constituent are sometimes exaggerated in the attached drawings. To facilitate understanding of explanation, in a cross-sectional view of each constituent in the attached drawings, illustration of portions other than cross sections, hatching, and the like are sometimes omitted.

### First Embodiment

FIG. 1 is a schematic perspective view of a configuration example of a temperature measurement device 1 according to a first embodiment of the present disclosure. The temperature measurement device 1 includes a tubular shaft 10, a temperature sensor unit 100, and a balloon 20. The shaft 10 is an example of a “tube” of the present disclosure, and the balloon 20 is an example of an “expansion member” of the present disclosure. In FIG. 1, an imaginary axis C indicating the axis of the shaft 10 is illustrated for convenience of explanation.

In the present specification, the direction parallel to the axis C is referred to as the axial direction, a direction perpendicular to the axis C is referred to as a radial direction, and the direction of the circumference of a circle centered on the axis C is referred to as a circumferential direction. As for the axial direction, the rightward direction on the drawing plane in FIG. 1 is defined as the positive direction. The positive axial direction is referred to as the distal direction or the leading-end portion side, and the negative axial direction is referred to as the proximal direction or the base-end portion side. As for a radial direction, a direction away from the axis C is sometimes referred to as an outward direction, and a direction toward the axis C is sometimes referred to as an inward direction.

The shaft 10 is, for example, a flexible tube such as the shaft of a catheter. The shaft 10 has the distal end (the leading end) 11 and the proximal end (the base end) 12. The shaft 10 is constructed to be inserted into a tubular organ in a living body such as the esophagus. For example, the distal end 11 of the shaft 10 is inserted into the mouth or the nose and then moved to the esophagus.

The temperature sensor unit 100 has a flexible sheet shape and is constructed to be stored in the shaft 10 in a stored state illustrated in FIG. 1.

In this specification, “flexibility” denotes, for example, a property of being bent by an external force. Flexibility may include elasticity and stiffness. For example, low stiffness is sometimes expressed as high flexibility. In this specification, “suppleness” includes flexibility. Suppleness may include such a property that an object can be freely deformed, in addition to flexibility.

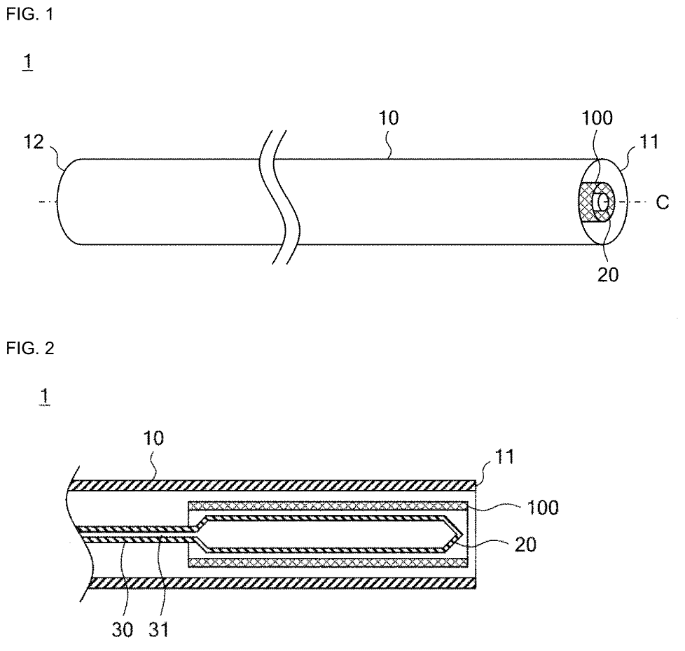

In the present embodiment, the temperature sensor unit 100 in the stored state is located radially between the shaft 10 and the balloon 20. The temperature sensor unit 100 can transition between the stored state in which the temperature sensor unit 100 is stored in the shaft 10 and an deployed state in which the temperature sensor unit 100 is deployed outward from the stored state. The following describes the stored state and the deployed state of the temperature sensor unit 100 with reference to FIGS. 2 to 5.

FIG. 2 is a cross-sectional view of the temperature measurement device 1 schematically illustrating an example of the stored state of the temperature sensor unit 100. The cross section illustrated in FIG. 2 shows a plane including the axis C. The proximal end of the balloon 20 is connected to a guide member 30 such as a wire. The proximal end of the guide member 30 extends to the outside through the proximal end 12 of the shaft 10. Alternatively, the proximal end of the guide member 30 may extend to the outside through an opening formed in the surface of the shaft 10. The user can operate a portion of the guide member 30 extending to the outside by using the user's hand, a driving device, or the like to move the balloon 20 connected to the guide member 30 in the axial direction. A portion of the temperature sensor unit 100 is connected to the balloon 20 by gluing or the like, so that when the balloon 20 is moved by using the guide member 30, the temperature sensor unit 100 can be moved in the axial direction along with the movement of the balloon 20.

The movement of the guide member 30, the balloon 20, and the temperature sensor unit 100 as mentioned above can be performed independently of the shaft 10. Hence, the guide member 30, the balloon 20, and the temperature sensor unit 100 can be moved in the axial direction relative to the shaft 10 and can be further moved beyond the distal end 11 of the shaft 10 in the distal direction to the outside of the shaft 10.

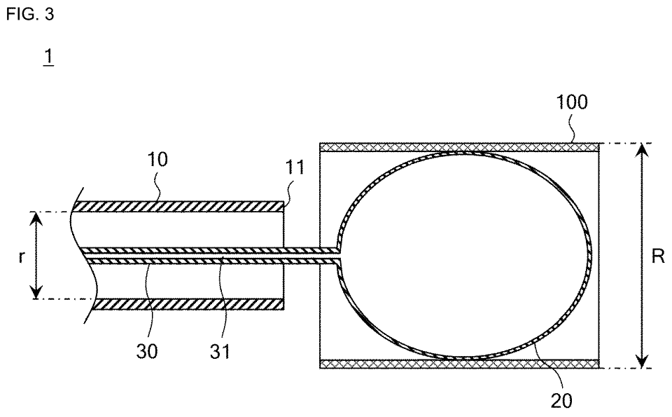

The balloon 20 can be reversibly deformed between a contracted state and an expanded state by introducing and removing a gas through a gas flow path 31 by using a pump or the like. Although the gas flow path 31 is located inside the guide member 30 in FIGS. 2 and 3, the gas flow path 31 may be provided separately from the guide member 30.

The balloon 20 is pushed out of the shaft 10 by the guide member 30 and then expanded radially outward into the expanded state so as to push and expand the temperature sensor unit 100. With this movement, the temperature sensor unit 100 transitions from the stored state illustrated in FIG. 2 to the deployed state illustrated in FIG. 3.

FIG. 3 is a cross-sectional view of the temperature measurement device 1 schematically illustrating an example of the deployed state of the temperature sensor unit 100. The balloon 20 and the temperature sensor unit 100 are positioned outside the shaft 10 in FIG. 3 as compared with those in the state illustrated in FIG. 2. In FIG. 3, the balloon 20 is inflated and in the expanded state, and the temperature sensor unit 100 is pushed and expanded by the balloon 20 and in the deployed state. In the deployed state, at least one radial dimension R of the temperature sensor unit 100 is larger than the inner diameter r of the shaft 10.

This configuration enables the temperature sensor unit 100 in the deployed state to be in contact with the inner wall of a tubular organ such as the esophagus in a living body.

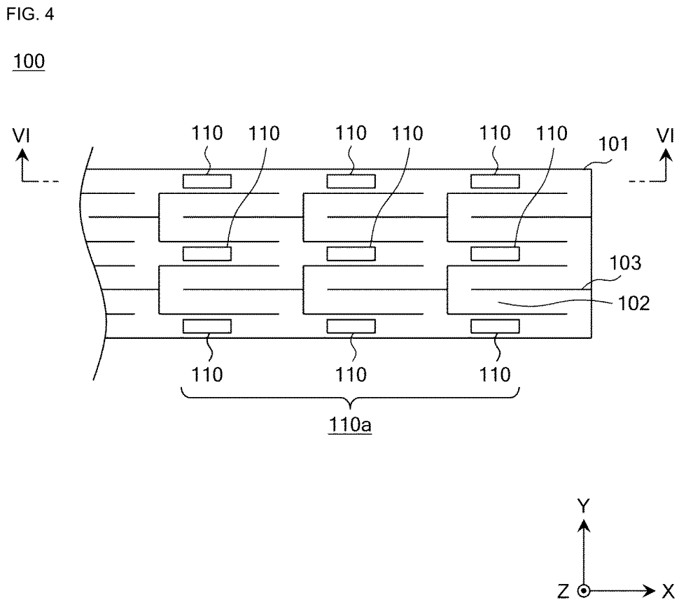

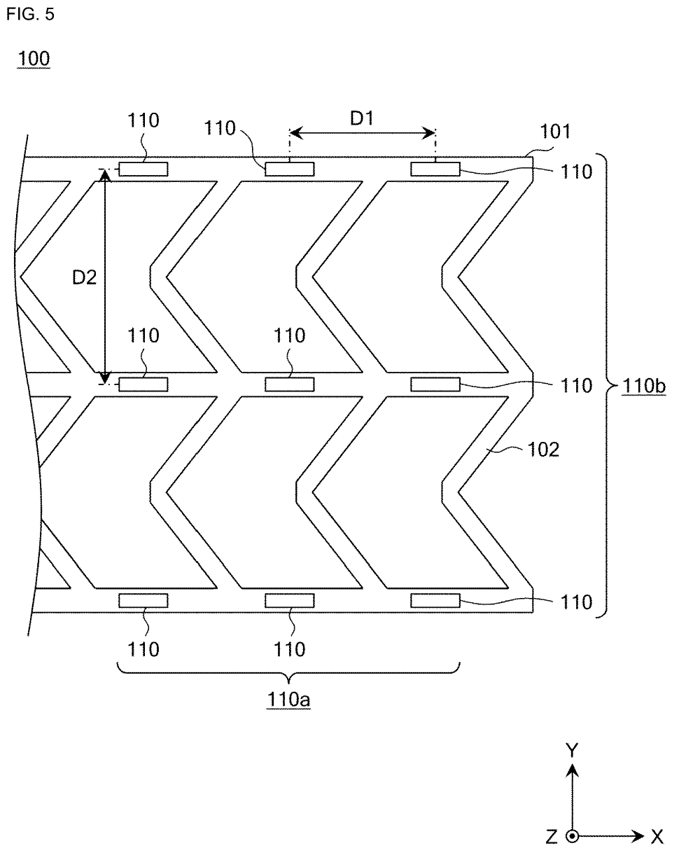

FIGS. 4 and 5 are schematic diagrams illustrating a configuration example of the temperature sensor unit 100. FIGS. 4 and 5 have the X-axis, the Y-axis, and the Z-axis orthogonal to one another for convenience of explanation. In the present specification, the X-axis direction is sometimes referred to as the row direction, and the Y-axis direction as the column direction. In the present embodiment, the X-axis direction corresponds to the axis C direction in FIG. 1, and the Z-axis direction correspond to the radial directions.

FIG. 4 schematically illustrates the temperature sensor unit 100 in the stored state. The temperature sensor unit 100 includes a sheet 101 and a plurality of temperature sensor elements 110 located on the sheet 101.

The sheet 101 is flexible and has a shape extending in the X direction and the Y direction. In FIG. 4, the Y direction corresponds to the circumferential direction. The sheet 101 has a tubular shape, which is omitted in FIG. 4, as a whole such that the sheet 101 further extends in the Y direction in FIG. 4, and that the upper end and the lower end of the sheet 101 in FIG. 4 are connected to each other. The sheet 101 contains, for example, polyimide, a liquid crystal polymer, polyethylene terephthalate, silicone, polyurethane, a polyether block amide, or a combination of some of these.

The temperature sensor elements 110 are sensors that output results of measurement of surrounding temperature. Each temperature sensor element 110 is, for example, a sensor such as a thermistor, a thermocouple, and a semiconductor temperature sensor. The temperature sensor elements 110 are connected to a control device with wiring interposed therebetween and constructed to transmit information indicating measurement results to the control device.

As for the X direction in FIG. 4, the temperature sensor elements 110 are arranged at equal intervals in the X direction. A plurality of temperature sensor elements 110 aligned at equal intervals in the X direction compose a row sensor-element set 110a. A plurality of row sensor-element sets 110a are arranged in the Y direction. FIG. 4 illustrates three row sensor-element sets 110a.

The region of the sheet 101 in which a row sensor-element set 110a is located and the region of the sheet 101 in which another row sensor-element set 110a adjacent to the row sensor-element set 110a is located are connected to each other with curved or bent arm portions 102. In the present embodiment, the arm portions 102 are part of the sheet 101. In the present embodiment, each arm portions 102 is formed by forming cuts 103 extending through the sheet 101 at certain portions of the sheet 101. The arm portion 102 is capable of expanding and contracting in the Y direction, so that when the temperature sensor unit 100 is pushed outward by the balloon 20, the arm portions 102 are stretched, and the temperature sensor unit 100 transitions from the stored state in FIG. 4 to the deployed state in FIG. 5.

FIG. 5 schematically illustrates the temperature sensor unit 100 in the deployed state. The curved or bent arm portions 102 in the stored state in FIG. 4 are extended in the deployed state in FIG. 5. With this configuration, the distance (D2 described later) between temperature sensor elements 110 adjacent in the Y direction in the deployed state in FIG. 5 is longer than in the stored state in FIG. 4. For example, for an application for the human esophagus, the height (the dimension in the X direction) of the tubular sheet 101 in the deployed state is in a range of 1 cm to 10 cm, for example, 6 cm, and the diameter of the sheet 101 is in a range of 1 cm to 5 cm, for example, 2 cm.

As for the Y direction in FIG. 5, the temperature sensor elements 110 in the deployed state are arranged at equal intervals in the Y direction. A plurality of temperature sensor elements 110 aligned at equal intervals in the Y direction compose a column sensor-element set 110b. A plurality of column sensor-element sets 110b are arranged in the X direction. FIG. 5 illustrates three column sensor-element sets 110b.

In the deployed state illustrated in FIG. 5, the distance (a first distance) between temperature sensor elements 110 adjacent in the X direction is D1, and the distance (a second distance) between temperature sensor elements 110 adjacent in the Y direction is D2. Specifically, in the deployed state, the temperature sensor elements 110 in the row sensor-element set 110a are aligned in the X direction at intervals of the first distance D1, and the temperature sensor elements 110 in the column sensor-element set 110b are aligned in the Y direction at intervals of the second distance D2.

The first distance D1 and the second distance D2 are determined depending on the application. In an application to monitor the temperature inside the esophagus to avoid thermal damage during left atrial ablation, for example, the first distance D1 and the second distance D2 are set within a range of 1 mm to 10 mm, for example, 6 mm. This setting enables the temperature inside the esophagus to be monitored at a resolution of specified intervals. In the case in which a monitored temperature exceeds a specified value, for example, the ablation is cancelled to prevent the heat from damaging biological tissue.

In a living body, heat tends to diffuse in the direction in which body fluid flows. Since blood vessels around the esophagus run along the esophagus, the heat applied to tissues around the esophagus, such as the heart, tends to diffuse in the direction in which the esophagus extends. Hence, in the direction in which the esophagus extends, unless temperature is monitored at short intervals and at high density (at high resolution), it is impossible to accurately detect the position at which the temperature has become high because of heat diffusion. Hence, in the present embodiment, the first distance D1 in the X direction corresponding to the extending direction of the esophagus in use may be constructed to be shorter than the second distance D2. For example, in a possible configuration, the first distance D1 is 1 mm or more and less than 6 mm, and the second distance D2 is 6 mm.

In this configuration, the temperature sensor elements 110 are arranged in the X direction, corresponding to the extending direction of the esophagus in use, at high density in such a degree that the position of the inner surface of the esophagus at which the temperature has become high can be accurately detected. As described above, the temperature sensor unit 100 is capable of accurately detecting an increase in the temperature of a tissue due to ablation in view of anatomical knowledge so that thermal damage to the tissue does not occur.

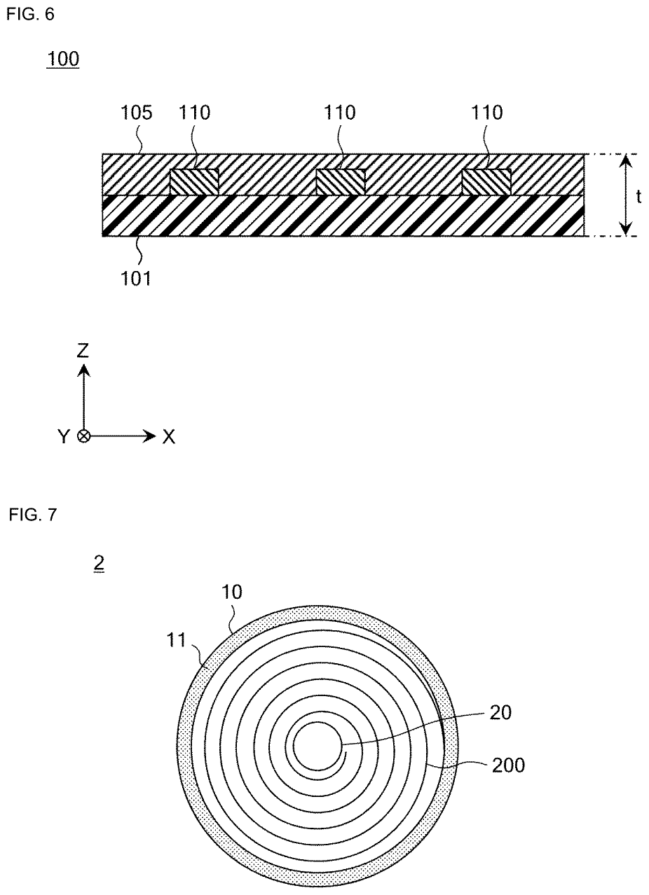

FIG. 6 is a schematic cross-sectional view of the temperature sensor unit 100 taken along line VI-VI in FIG. 4. As described earlier, the plurality of temperature sensor elements 110 are located on the sheet 101. The temperature sensor unit 100 may include a protective layer 105 covering the temperature sensor elements 110. This makes it possible to alleviate external shocks and prevent the temperature sensor elements 110 from being damaged. The protective layer 105 also prevents the temperature sensor elements 110, wiring, and the like from coming into contact with water and deteriorating.

In the example illustrated in FIG. 6, although the protective layer 105 covers the entire surface of the plurality of temperature sensor elements 110 and the sheet 101, the present embodiment is not limited to this configuration. For example, the protective layer 105 may be located only on the one or more temperature sensor elements 110. Alternatively, the protective layer 105 may cover the upper surfaces and the side surfaces of the one or more temperature sensor elements 110.

The protective layer 105 contains, for example, polyimide, a liquid crystal polymer, polyethylene terephthalate, silicone, polyurethane, a polyether block amide, or a combination of some of these.

The protective layer 105 may contain, for example, a metal such as Cu, Al, Ni, Ag, and Au. In the case in which the protective layer 105 contains a metal having high thermal conductivity, external heat, for example, heat of the inner surface of the esophagus, is quickly transmitted to the temperature sensor elements 110. Thus, the temperature measurement device 1 can measure external temperature with high accuracy.

The thickness t of the temperature sensor unit 100 is, for example, 1 mm or less. Such a small thickness enables the temperature sensor unit 100 to be flexible and to be deformed conforming to the shape of the inner wall of an organ. Thus, each of the temperature sensor elements 110 can be in close contact with the inner wall of the organ, which makes it possible to accurately measure the temperature inside the organ. The thickness t of the temperature sensor unit 100 is not limited to these numerical values and may be 0.5 mm or less or may be 0.1 mm or less.

Since the temperature sensor unit 100 is thin as described above, the heat capacity of the temperature sensor unit 100 can be small. Accordingly, the thermal response of the temperature sensor unit 100 is fast, which makes it possible to accurately measure the temperature inside the organ.

The thickness t of the temperature sensor unit 100 is expressed as, for example, the sum of the thickness of the sheet 101 and the thickness of the protective layer 105. The protective layer 105 is not essential to the temperature sensor unit 100. In a configuration without the protective layer 105, the thickness t of the temperature sensor unit 100 may be expressed as, for example, the sum of the thickness of the sheet 101 and the thickness of the temperature sensor element 110.

The temperature sensor unit 100 may further include a metal layer composed of a metal such as Cu, Al, Ni, Ag, and Au. The metal layer is located, for example, on the sheet 101 or between the sheet 101 and the protective layer 105 and used for wiring for the temperature sensor elements 110. The metal layer increases the strength of the temperature sensor unit 100 against shocks, bending, and like.

The temperature measurement device 1 as described above is used, for example, by users such as doctors as described below.

(1) The user inserts the shaft 10 through the nose and/or the mouth into the esophagus and places the temperature measurement device 1 including the temperature sensor unit 100 in the stored state and the balloon 20 in the contracted state (see FIGS. 1 and 2) in the esophagus.

(2) The user pushes the guide member 30 in the distal direction with the shaft 10 fixed to move the balloon 20 and the temperature sensor unit 100 through the distal end 11 of the shaft 10 to the outside of the shaft 10.

(3) The user sends a gas to the balloon 20 by using a pump or the like to put the balloon 20 into the expanded state (see FIG. 3).

(4) The user obtains the measurement results of the plurality of temperature sensor elements 110 of the temperature sensor unit 100.

Instead of the above (2), the user may pull the shaft 10 with the guide member 30 fixed to move the balloon 20 and the temperature sensor unit 100 through the distal end 11 of the shaft 10 to the outside of the shaft 10.

As described above, the temperature measurement device 1 according to the present embodiment includes the shaft 10 which is an example of a tube and the temperature sensor unit 100. The temperature sensor unit 100 includes the flexible sheet 101 and the plurality of temperature sensor elements 110 located on the sheet 101 and is constructed to be stored in the shaft 10.

With this configuration, since the sheet 101 is flexible, the temperature sensor unit 100 can be deformed conforming to the inner wall of the shaft 10. Hence, gaps are less likely to occur in the shaft 10, which enables the temperature sensor elements 110 to be arranged at high density in the shaft 10.

In addition, since the sheet 101 of the temperature sensor unit 100 is flexible, the sheet 101 comes into close contact easily with the inner wall of an organ containing water due to surface tension. Hence, the temperature sensor unit 100 can measure the temperature inside the organ with higher accuracy than conventional techniques.

The temperature sensor unit 100 may be constructed to transition between the stored state in which the temperature sensor unit 100 is stored in the shaft 10 and the deployed state in which the temperature sensor unit 100 is deployed outside the shaft 10.

With this configuration, since the temperature sensor unit 100 stored at high density in the shaft 10 is deployed, it is possible to measure the temperature in a wide area.

The shaft 10 may have the distal end 11 and the proximal end 12. The temperature measurement device 1 may further include the guide member 30 that moves the temperature sensor unit 100 from the inside of the shaft 10 through the distal end 11 to the outside of the shaft 10. The temperature sensor unit 100 may be constructed such that after the temperature sensor unit 100 in the stored state is moved through the distal end 11 to the outside of the shaft 10 by the guide member 30, the temperature sensor unit 100 is deployed in the directions from the inside toward the outside of the shaft 10 in cross-sectional view of the shaft 10 in a direction intersecting the direction from the distal end 11 to the proximal end 12 and is thus deformed into the deployed state.

With this configuration, since the temperature sensor unit 100 stored at high density in the shaft 10 is deployed, it is possible to measure the temperature inside a tubular organ of a living body in a wide area.

The temperature measurement device 1 may further include the balloon 20 which is an example of an expansion member. The balloon 20 can be expanded in the directions from the inside toward the outside of the shaft 10 in cross-sectional view of the shaft 10 in a direction intersecting the direction from the distal end 11 to the proximal end 12 and can apply pressure to the temperature sensor unit 100 in the directions from the inside toward the outside of the shaft 10 in cross-sectional view of the shaft 10.

With this configuration, the temperature sensor unit 100 stored at high density in the shaft 10 can be deployed.

At least part of the balloon 20 may be located radially inside the temperature sensor unit 100 in the stored state.

With this configuration, the temperature sensor unit 100 comes into direct contact with the inner wall of an organ more easily and thus can measure the temperature inside an organ with higher accuracy.

The temperature measurement device 1 may further include the protective layer 105 covering the temperature sensor unit 100.

With this configuration, the protective layer 105 alleviates external shocks and prevents the temperature sensor elements 110 from being damaged. The protective layer 105 also prevents constituents such as the temperature sensor unit 100 and wiring from coming into contact with water and deteriorating.

The protective layer 105 may contain a metal.

This configuration enables external heat, for example, heat of the inner surface of the esophagus, to be transmitted quicky to the temperature sensor unit 100 through the protective layer 105 containing a metal. Thus, the temperature measurement device 1 can measure external temperature with high accuracy.

The plurality of temperature sensor elements 110 are arranged such that the distance between adjacent temperature sensor elements 110 of the plurality of temperature sensor elements 110 is smaller than or equal to a specified value.

This configuration enables the temperature inside an organ to be measured at a plurality of points.

The temperature sensor unit 100 may include four or more temperature sensor elements 110. In this case, the four or more temperature sensor elements 110 in the deployed state compose the row sensor-element sets 110a and the column sensor-element sets 110b each including two or more temperature sensor elements 110 aligned in a row direction and a column direction, respectively, on the temperature sensor unit 100, the row direction and the column direction intersecting each other. The temperature sensor elements 110 in the row sensor-element set 110a are aligned at intervals of the first distance D1 in the axial direction of the shaft 10 in the deployed state. The temperature sensor elements 110 in the column sensor-element set 110b are aligned at intervals of the second distance D2 in a direction intersecting the axial direction of the shaft 10 in the deployed state. The first distance D1 is shorter than the second distance D2.

In this configuration, the temperature sensor elements 110 are located at higher density in the row direction corresponding to the extending direction of a tubular organ in use than in the column direction. Accordingly, the temperature measurement device 1 can measure the temperature in the row direction with high accuracy.

### Second Embodiment

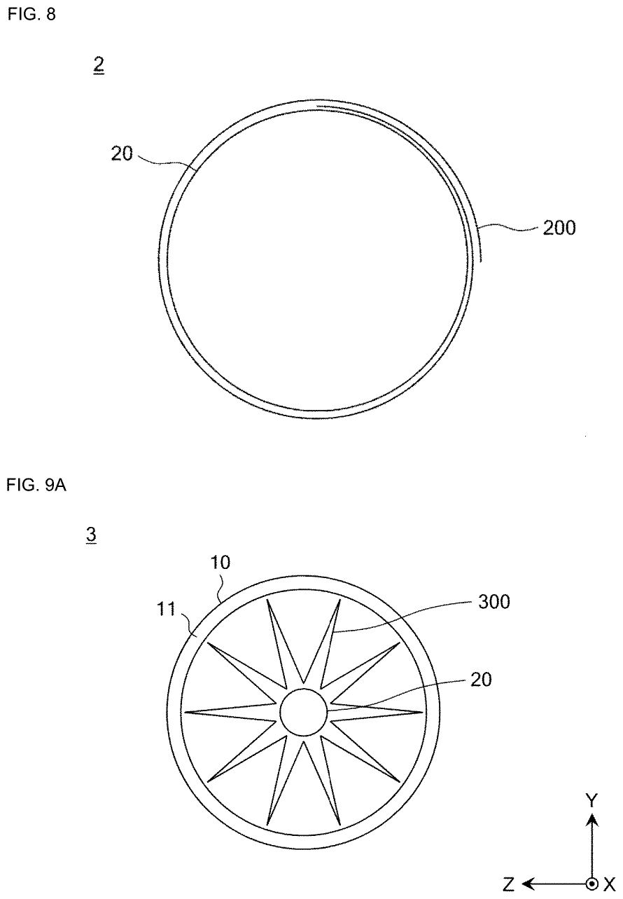

FIGS. 7 and 8 are schematic diagrams illustrating a configuration example of a temperature sensor unit 200 in a temperature measurement device 2 according to a second embodiment of the present disclosure. FIG. 7 is a schematic plan view of the temperature sensor unit 200 in a stored state, from the distal side of the axis C (the right side in the drawing plane in FIG. 1). In FIG. 7, the distal end 11 of the shaft 10 is hatched with dots to clearly distinguish the constituents.

As illustrated in FIG. 7, the temperature sensor unit 200, which is flexible and sheet-shaped, is rolled around the balloon 20 in the stored state. This configuration enables the radial dimension of the temperature sensor unit 200 to be small, so that the temperature sensor unit 200 can be stored in the shaft 10.

As in the first embodiment, after the balloon 20 is pushed out of the shaft 10 in the distal direction, the balloon 20 is put in an expanded state, so that the temperature sensor unit 200 is pushed outward and expanded. With this operation, the temperature sensor unit 200 transitions from the stored state illustrated in FIG. 7 to a deployed state illustrated in FIG. 8.

FIG. 8 is a schematic plan view of the temperature sensor unit 200 in the deployed state, from the distal side of the axis C. The temperature sensor unit 200 rolled around the balloon 20 is unrolled, as the balloon 20 is expanded, and pushed outward and expanded by the balloon 20. In the deployed state in FIG. 8, the radial dimension of the temperature sensor unit 200 has become larger than in the stored state illustrated in FIG. 7. With this configuration, the temperature sensor unit 200 in the deployed state can be in contact with the inner wall of a tubular organ such as the esophagus in a living body.

In the present embodiment, the surface of the sheet of the temperature sensor unit 200 is hydrophilic. In particular, the surface of the sheet constructed to face the inner wall of a tubular organ in a living body, into which the temperature sensor unit 100 is inserted, in the deployed state of the temperature sensor unit 100 is hydrophilic. For example, the surface of the sheet is formed of a hydrophilic material to be hydrophilic. Alternatively, the surface of the sheet may be hydrophilic by being processed to be hydrophilic.

In this specification, “hydrophilic” denotes such a property of a target surface that when the contact angle θ between water and the target surface (the surface of the sheet in the present embodiment) is measured according to the method indicated in the following (1) to (3), 0 degrees<θ≤90 degrees.

(1) The temperature sensor unit 100 is placed such that the target surface faces upward and is level.

(2) A water droplet is dropped onto the target surface and left to stand for a specified time (for example, 10 minutes).

(3) The contact angle θ between the water and the target surface is measured.

Since the surface of the sheet of the temperature sensor unit 200 is hydrophilic, the layers of the sheet come into close contact easily with one another when the temperature sensor unit 200 is put into the stored state, which enables the temperature sensor unit 200 to be stored at high density in the shaft 10.

### Third Embodiment

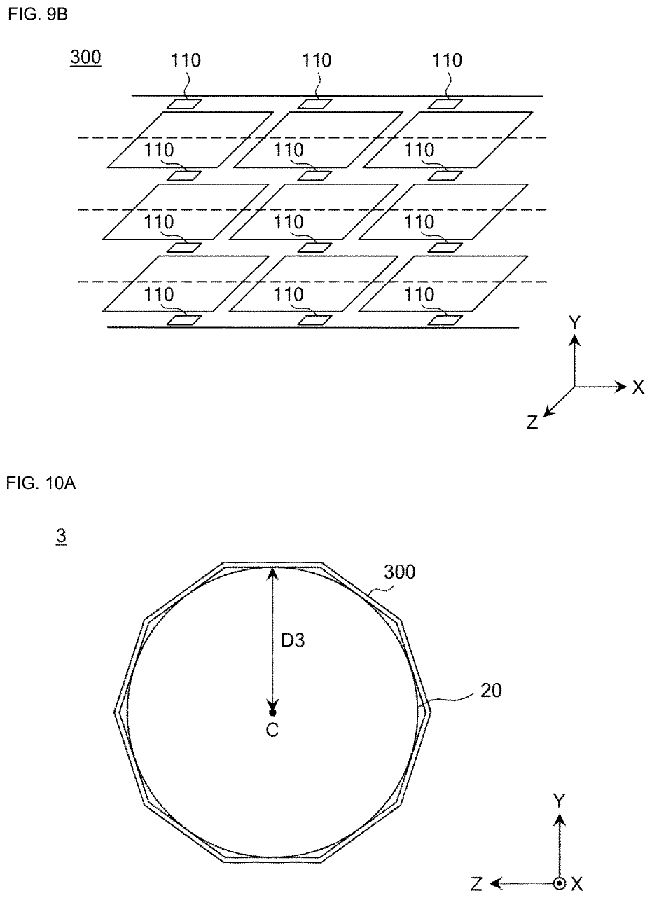

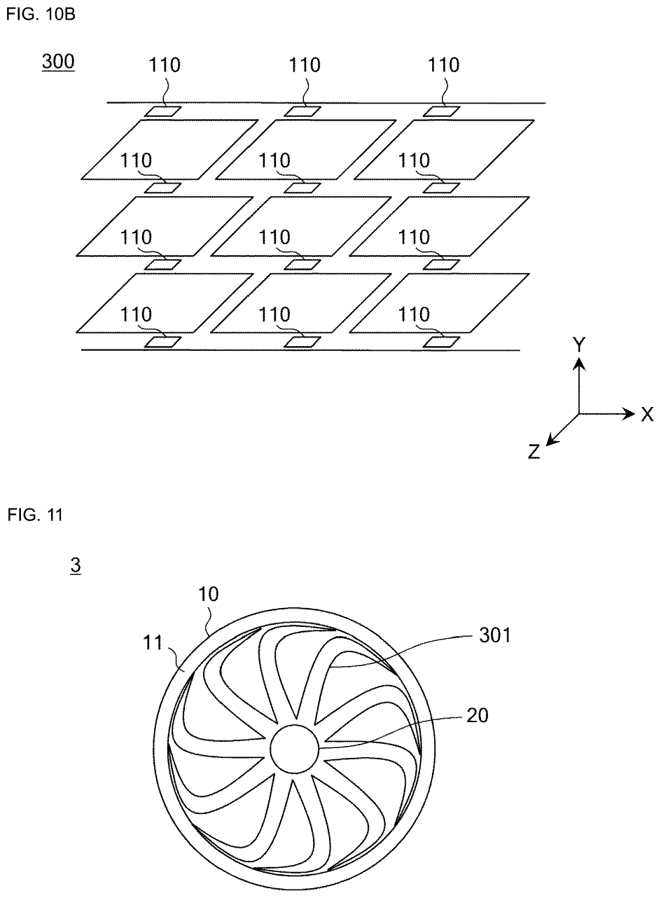

FIGS. 9A, 9B, 10A, and 10B are schematic diagrams illustrating a configuration example of a temperature sensor unit 300 in a temperature measurement device 3 according to a third embodiment of the present disclosure. FIG. 9A is a schematic plan view of the temperature sensor unit 300 in a stored state, from the distal side of the axis C.

As illustrated in FIG. 9A, in the stored state, the temperature sensor unit 300 which is flexible and sheet-shaped has a plurality of creases extending in the axial direction and is bent along these creases and folded. This configuration enables the radial dimension of the temperature sensor unit 300 to be small, so that the temperature sensor unit 300 can be stored in the shaft 10.

FIG. 9B is a schematic perspective view of the temperature sensor unit 300 in the stored state. The creases of the temperature sensor unit 300 are formed, for example, at the positions indicated by the dashed lines in FIG. 9B. The temperature sensor elements 110 are arranged not to be located over the creases.

As in the first and second embodiments, after the balloon 20 is pushed out of the shaft 10 in the distal direction, the balloon 20 is put into the expanded state, so that the temperature sensor unit 300 is pushed outward and expanded. With this operation, the temperature sensor unit 300 transitions from the stored state illustrated in FIG. 9A to a deployed state illustrated in FIG. 10A.

FIG. 10A is a schematic plan view of the temperature sensor unit 300 in the deployed state, from the distal side of the axis C. The folded temperature sensor unit 300 is pushed outward and expanded by the expanding balloon 20. With this operation, the radial dimension of the temperature sensor unit 300 has become larger in the deployed state in FIG. 10A than in the stored state illustrated in FIG. 9A.

FIG. 10B is a schematic perspective view of the temperature sensor unit 300 in the deployed state. The creases of the temperature sensor unit 300 are unfolded in the deployed state. Thus, the temperature sensor unit 300 transitions to the deployed state by the creases being unfolded.

FIG. 11 is a schematic diagram illustrating a temperature sensor unit 301 according to a modification example of the present embodiment. FIG. 11 is a schematic plan view of the temperature sensor unit 301 in a stored state, from the distal side of the axis C. As illustrated in FIG. 11, portions of the temperature sensor unit 301 in the stored state may be in contact with the inner wall of the shaft 10 and be bent conforming to the inner wall of the shaft 10. This configuration enables the minimum distance between the axis C and the temperature sensor unit 301 in the deployed state to be larger than the minimum distance D3 between the axis C and the temperature sensor unit 300 illustrated in FIG. 10.

### Fourth Embodiment

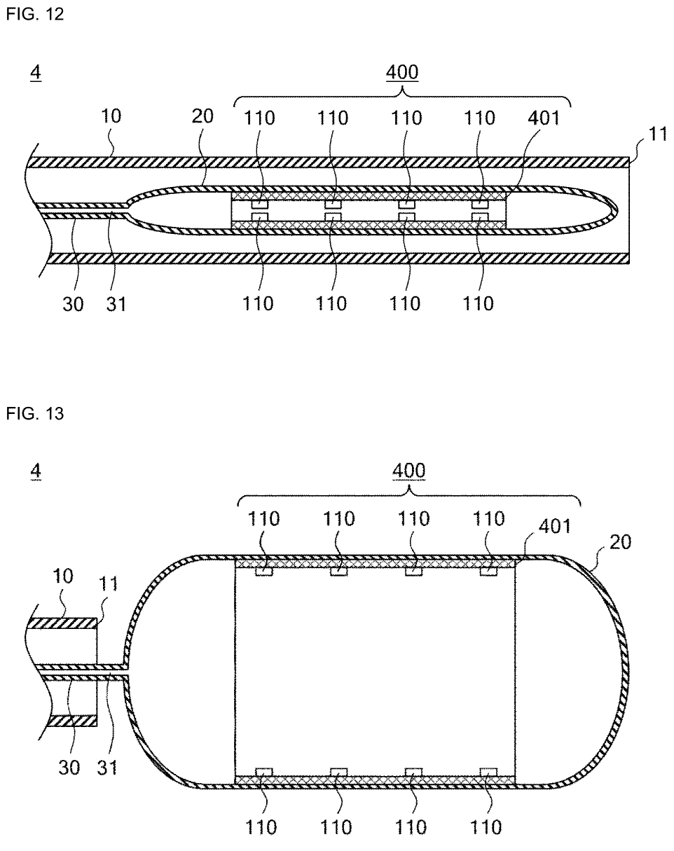

FIGS. 12 and 13 are schematic cross-sectional views of a configuration example of a temperature measurement device 4 according to a fourth embodiment of the present disclosure. In contrast to the first embodiment in which the temperature sensor unit 200 are located outside the balloon 20 (for example, see FIGS. 2 and 3), in the present embodiment, at least part of the balloon 20 is located radially outside a temperature sensor unit 400 in the stored state. In other words, the temperature sensor unit 400 of the temperature measurement device 4 is located inside the balloon 20.

FIG. 12 schematically illustrates the temperature sensor unit 400 in the stored state. FIG. 13 schematically illustrates the temperature sensor unit 400 in the deployed state. At least part of the temperature sensor unit 400 is physically connected to the inner surface of the balloon 20 by a method such as gluing. In this configuration, after the balloon 20 is pushed out of the shaft 10 by the guide member 30, when the balloon 20 transitions from a contracted state to an expanded state, the balloon 20 pulls the temperature sensor unit 400 radially outward. The pulled temperature sensor unit transitions from the stored state to the deployed state.

In FIGS. 12 and 13, the plurality of temperature sensor elements 110 are located on the inner surface (the inner side portion) of a sheet 401. However, the present embodiment is not limited to this configuration. The plurality of temperature sensor elements 110 may be located on the outer surface (the outer side portion) of the sheet 401. In other words, the plurality of temperature sensor elements 110 may be located between the outer surface of the sheet 401 and the inner surface of the balloon 20. Alternatively, the plurality of temperature sensor elements 110 may be located on both the outer side portion and the inner side portion of the sheet 401.

As described above, since the temperature sensor unit 400 is located inside the balloon 20 in the present embodiment, the temperature sensor unit 400 follows the movement of the balloon 20, and thus the removal of the temperature sensor unit 400 can be performed safely and easily.

### Fifth Embodiment

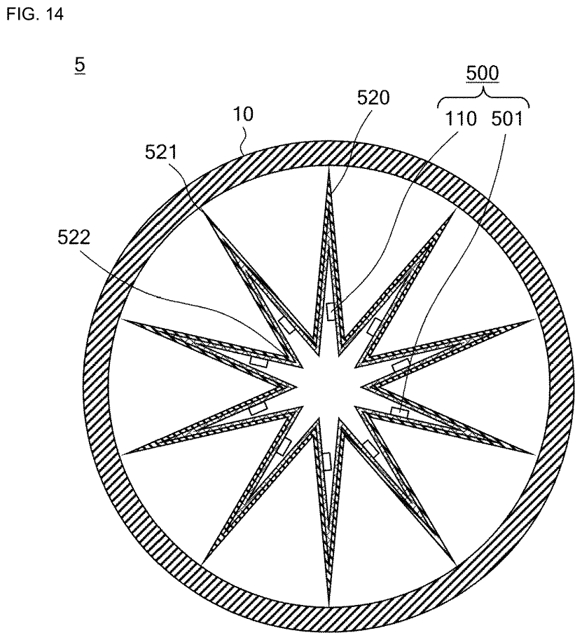

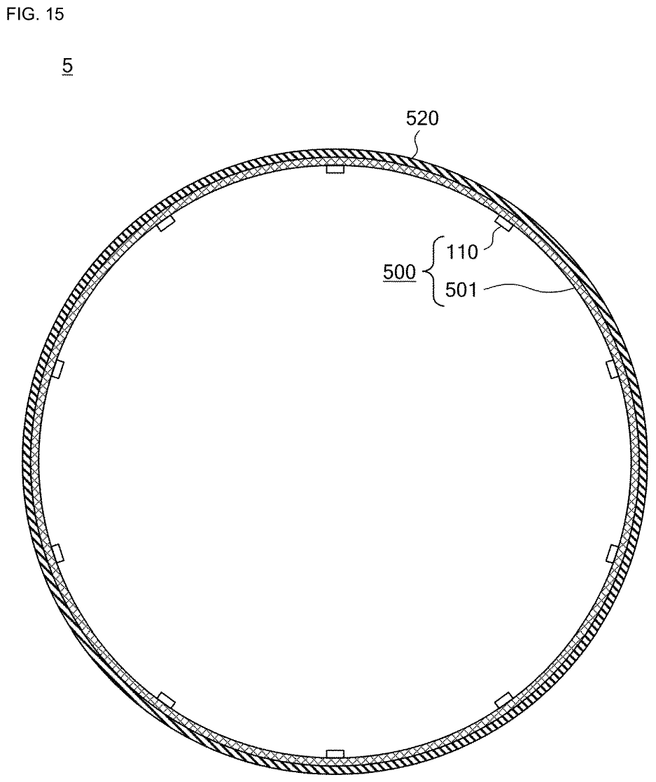

FIGS. 14 and 15 are schematic cross-sectional views of a configuration example of a temperature measurement device 5 according to a fifth embodiment of the present disclosure. FIG. 14 schematically illustrates a temperature sensor unit 500 of the temperature measurement device 5 in a stored state. FIG. 15 schematically illustrates the temperature sensor unit 500 in a deployed state.

A balloon 520 of the temperature measurement device 5 according to the present embodiment has creases 521 and 522 extending in the axial direction. As illustrated in FIG. 14, the balloon 520 in a contracted state is stored in the shaft 10, being bent along the creases 521 and 522. In the present specification, the creases 521 are sometimes referred to as mountain fold lines, and the creases 522 as valley fold lines. Although the example illustrated in FIG. 14 has ten creases 521 and ten creases 522, the number of creases is not limited to this configuration.

A sheet 501 of the temperature sensor unit 500 is located on the inner surface (the inner side portion) of the balloon 520. The plurality of temperature sensor elements 110 are located on the inner surface (the inner side portion) of the sheet 501. At least part of the temperature sensor unit 500 is physically connected to the inner surface of the balloon 520 by a method such as gluing. With this configuration, when the balloon 520 transitions from the contracted state to an expanded state, the temperature sensor unit 500 transitions from the stored state in FIG. 14 to the deployed state in FIG. 15.

When the balloon 520 is folded, the sheet 501 located on the balloon 520 is also folded. The sheet 501 may have creases at positions corresponding to those of the creases 521 and 522 of the balloon 520 which underlies the sheet 501.

The temperature sensor unit 500 is in contact with the balloon 520, and the plurality of temperature sensor elements 110 are arranged so as not to overlap the creases 521 and 522. For example, as illustrated in FIG. 14, the temperature sensor elements 110 are located on portions of the sheet 501 that are not bent when the balloon 520 is folded. In the case in which the sheet 501 has creases, the temperature sensor elements 110 are arranged on the sheet 501 so as not to be located over the creases. For example, the temperature sensor elements 110 are arranged on portions of the sheet 501 where the creases are not located.

Since the temperature sensor elements 110 are arranged on portions of the sheet 501 that are not bent, the size of the temperature sensor unit 500 when folded can be small, so that a larger number of temperature sensor elements 110 can be stored in the shaft 10. In addition, it is possible to prevent the temperature sensor elements 110 from receiving force such as bending stress.

### Modification Example of Fifth Embodiment

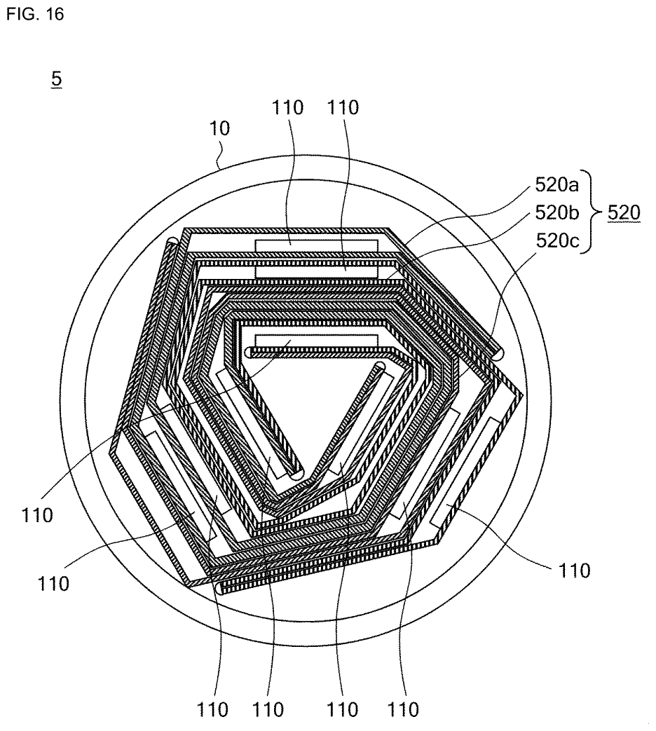

FIGS. 16 to 18 are schematic cross-sectional views of a configuration example of a temperature measurement device 5 according to a modification example of the fifth embodiment. FIG. 16 is a schematic plan view of a balloon 520 in a contracted state, from the distal side of the axis C. As illustrated in FIG. 16, the balloon 520 in the contracted state is bent along creases and stored in the shaft 10.

In the example illustrated in FIG. 16, the balloon 520 includes a first portion 520a, a second portion 520b, and a third portion 520c. In FIGS. 16 to 18, to facilitate understanding of explanation, the first portion 520a, the second portion 520b, and the third portion 520c are hatched differently. The first portion 520a, the second portion 520b, and the third portion 520c are connected to one another. Each of the first portion 520a, the second portion 520b, and the third portion 520c has creases extending in the axial direction and bent along the creases. This configuration enables the balloon 520 to be stored in the shaft 10.

A plurality of temperature sensor elements 110 are located on portions of the first portion 520a, the second portion 520b, and the third portion 520c of the balloon 520 where the creases are not located.

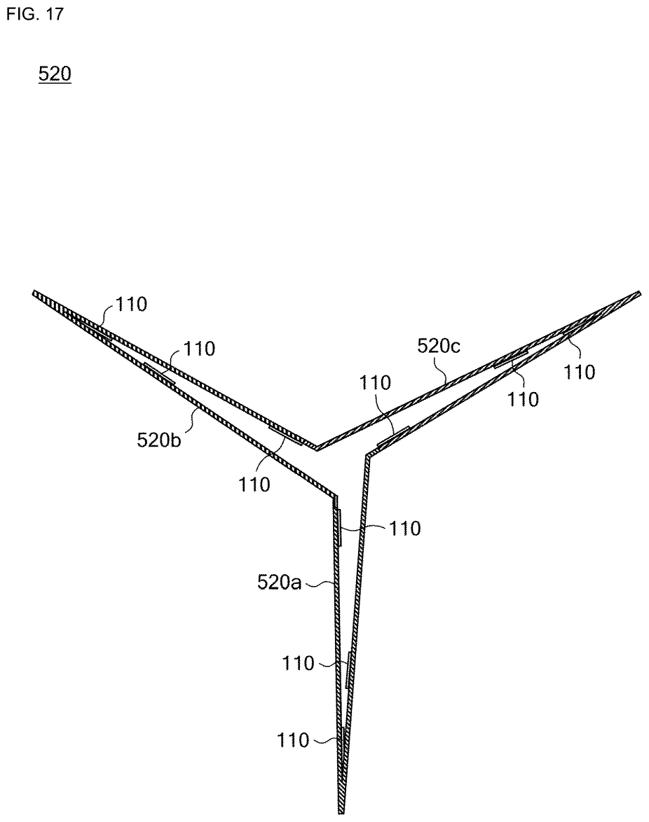

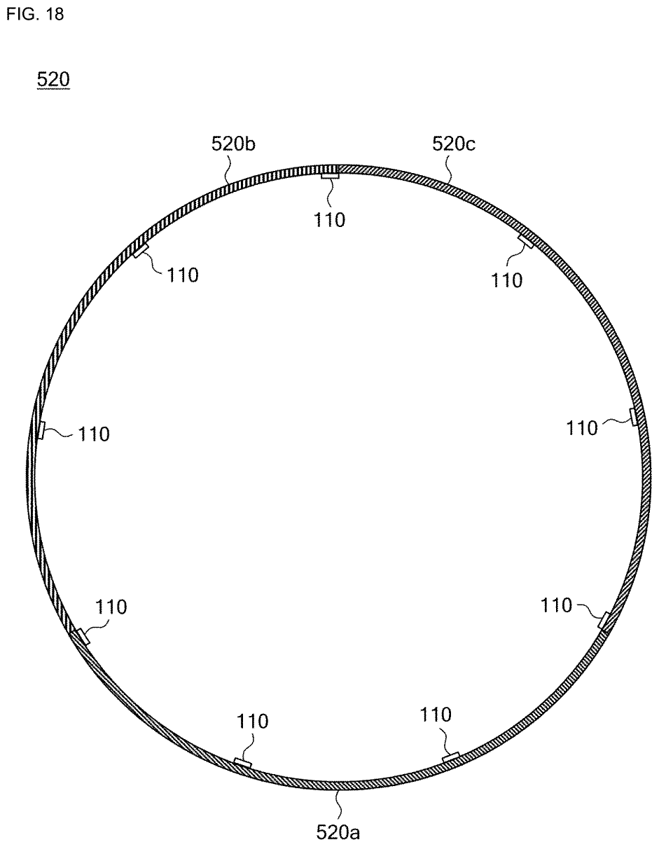

As illustrated in FIG. 17, when the balloon 520 is moved to the outside of the shaft 10, the first portion 520a, the second portion 520b, and the third portion 520c are unfolded along the creases, and the balloon 520 is radially expanded. When a gas is injected into the balloon 520 in an intermediate state illustrated in FIG. 17, the balloon 520 is inflated and transitions to an expanded state illustrated in FIG. 18. In the expanded state, the plurality of temperature sensor elements 110 are located, for example, at equal intervals in the circumferential direction.

### Sixth Embodiment

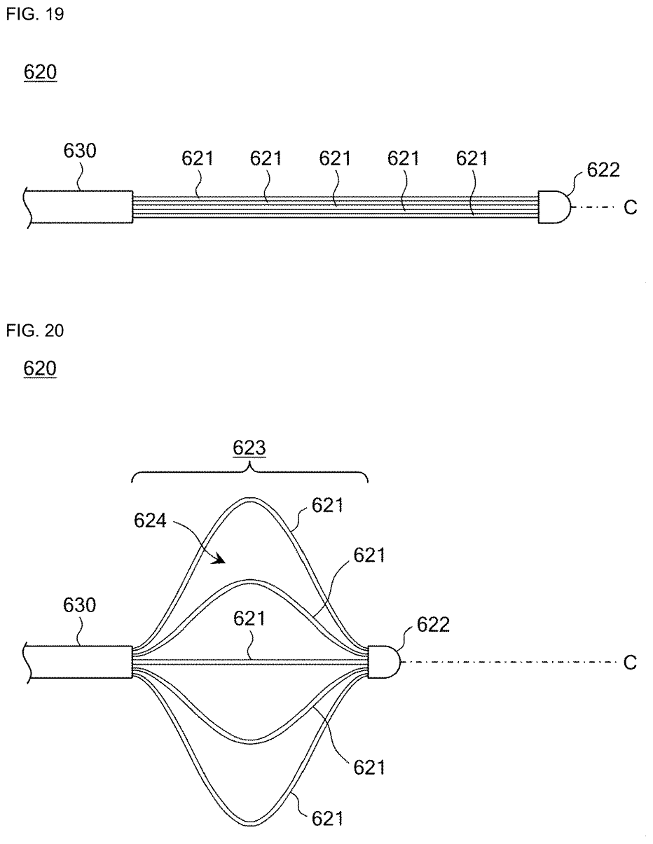

The following describes a temperature measurement device according to a sixth embodiment of the present disclosure with reference to FIGS. 19 to 22. The main difference between the first embodiment and the present embodiment is that the temperature measurement device 1 according to the first embodiment includes the balloon 20 as an expansion member, whereas the temperature measurement device according to the present embodiment includes a basket catheter 620 as an expansion member.

FIGS. 19 and 20 are schematic side views of a configuration example of the basket catheter 620 of the temperature measurement device according to the present embodiment. FIGS. 19 and 20 illustrate the basket catheter 620 in a contracted state and an expanded state, respectively.

The basket catheter 620 includes a cylindrical guide member 630 and a plurality of wires 621 each extending in the axial direction and constructed to be stored in the guide member 630. The distal end of each of the wires 621 is bound by, for example, a binding portion 622. Alternatively, the distal end of each of the wires 621 may be bound by a method such as gluing, fusing, or the like.

As illustrated in FIG. 20, the wires 621 are constructed to be curved and radially expanded and form a basket portion 623 having a basket shape surrounding a space 624. With this operation, the basket catheter 620 transitions to the expanded state. The basket catheter 620 is not limited to the example mentioned above and may employ a publicly-known configuration for a basket catheter.

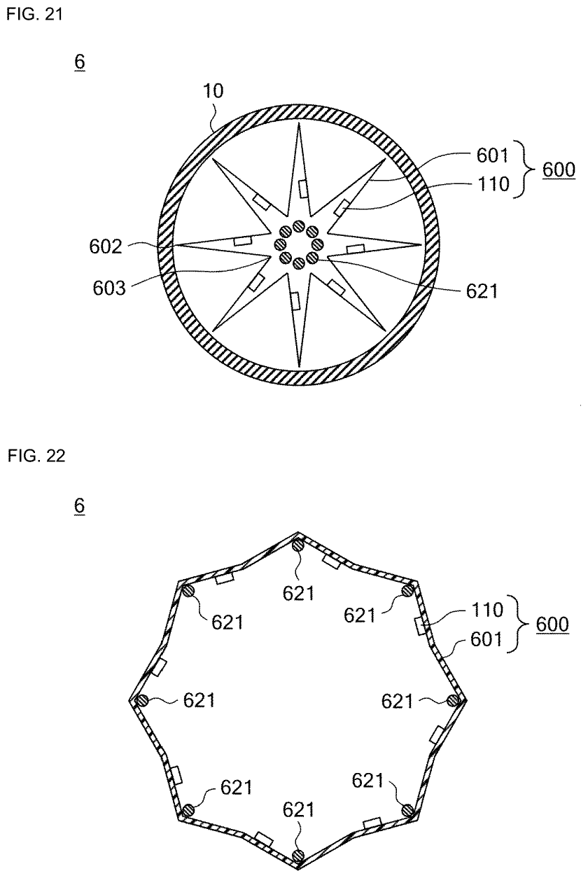

FIGS. 21 and 22 are schematic cross-sectional views of a configuration example of a temperature measurement device 6 according to the present embodiment. FIG. 21 schematically illustrates a temperature sensor unit 600 of the temperature measurement device 6 in a stored state. FIG. 22 schematically illustrates the temperature sensor unit 600 in a deployed state.

As illustrated in FIGS. 21 and 22, a sheet 601 of the temperature sensor unit 600 has creases 602 and 603 extending in the axial direction. As illustrated in FIG. 21, the sheet 601 in a contracted state is bent along the creases 602 and 603 and stored in the shaft 10.

A plurality of temperature sensor elements 110 are arranged on the sheet 601 so as not to be located over the creases 602 and 603. In the example illustrated in FIGS. 21 and 22, the plurality of temperature sensor elements 110 are located on portions of the inner surface of the sheet 601 where the creases 602 and 603 are not located. However, the present embodiment is not limited to this configuration. The plurality of temperature sensor elements 110 may be arranged on the outer surface of the sheet 601 or on both the inner surface and the outer surface.

After the basket catheter 620 in the contracted state illustrated in FIG. 21 is moved to the outside of the shaft 10, when the basket catheter 620 transitions to the expanded state, the sheet 601 of the temperature sensor unit 600 is pushed and expanded by the plurality of wires 621 of the basket catheter 620. With this operation, the temperature sensor unit 600 transitions from the stored state illustrated in FIG. 21 to the deployed state illustrated in FIG. 22.

### Seventh Embodiment

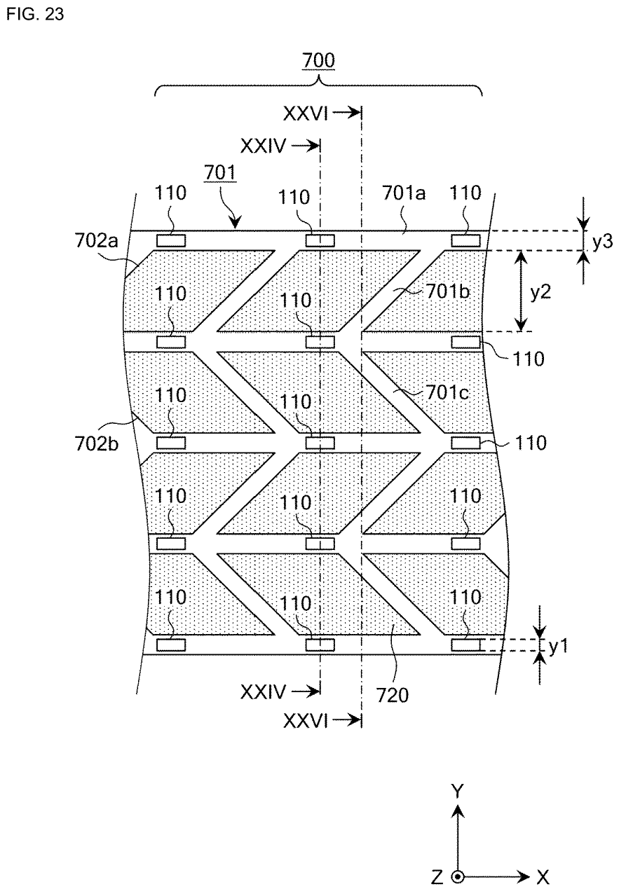

The following describes a temperature measurement device according to a seventh embodiment of the present disclosure with reference to FIGS. 23 to 30.

FIG. 23 is a schematic diagram illustrating a configuration example of a temperature sensor unit 700 and a balloon 720 of a temperature measurement device according to the present embodiment. FIG. 23 have the X-axis, the Y-axis, and the Z-axis orthogonal to one another for convenience of explanation. The direction of the X-axis is parallel to the extending direction of the shaft 10 (for example, the direction of the axis C in FIG. 1). Here, “the extending direction of the shaft 10” indicates the direction in which the shaft 10 extends, for example, when the shaft 10 is extended in a straight line. In FIG. 23, the balloon 720 is hatched with dots to facilitates understanding of the configuration example. Hence, the dot hatching in FIG. 23 is not intended to indicate a cross section of the balloon 720.

At least part of the temperature sensor unit 700 is in contact with the front surface or the back surface of the balloon 720. At least part of the temperature sensor unit 700 may be physically connected to the front surface or the back surface of the balloon 720 by a method such as gluing.

The balloon 720 and the temperature sensor unit 700 in a stored state are, for example, as illustrated in FIG. 14 or 16, bent along creases extending in the X direction and stored in the shaft 10. As mentioned above, when the balloon 720 and the temperature sensor unit 700 transition from a deployed state to the stored state, the balloon 720 and the temperature sensor unit 700 are constructed to be folded such that the dimension in the Y direction decreases.

The temperature sensor unit 700 includes a sheet 701 and a plurality of temperature sensor elements 110 located on the sheet 701. In the example illustrated in FIG. 23, the sheet 701 includes a plurality of first portions 701a extending in the X direction and second portions 701b connecting first portions 701a adjacent in the Y direction. The sheet 701 further includes third portions 701c extending from the side of the first portions 701a opposite to the second portions 701b in the Y direction. The third portions 701c connect first portions 701a adjacent in the Y direction.

In the example illustrated in FIG. 23, the sheet 701 includes a plurality of openings 702a surrounded by the first portions 701a and the second portions 701b and a plurality of openings 702b surrounded by the first portions 701a and the third portions 701c. As described above, the sheet 701 has a lattice shape including the plurality of line-shaped portions 701a, 701b, and 701c extending in different directions.

For example, the first portions 701a, the second portions 701b, and the third portions 701c of the sheet 701 have wiring for connecting a plurality of sensor elements 110 to one another.

In the present specification, the direction in which the second portions 701b or the third portions 701c of the sheet 701 extend is referred to as “first direction” in some cases. In the case in which the first direction mentioned above is the direction in which the second portions 701b of the sheet 701 extend, the direction in which the third portions 701c extend is referred to as “third direction” in some cases. Alternatively, in the case in which the first direction mentioned above is the direction in which the third portions 701c of the sheet 701 extend, the direction in which the second portions 701b extend is referred to as “third direction” in some cases.

The first direction is set to be not parallel to the Y direction (which is sometimes referred to as “third direction” or “folding direction” in this specification) and not orthogonal to the Y direction. In the example illustrated in FIG. 23, the angle φ2 of the second portion 701b relative to the +X direction satisfies the relation 0°<φ2<90° or 180°<φ2<270°. In addition, in the example illustrated in FIG. 23, the angle φ3 of the third portion 701c relative to the +X direction satisfies the relation 90°<φ3<180° or 270°<φ3<360°.

As described above, the first direction is set not to be parallel or orthogonal to the third direction (the Y direction). Since the X direction is orthogonal to the Y direction in the example illustrated in FIG. 23, the first direction is not parallel or orthogonal not only to the third direction (the Y direction) but also to the extending direction of the shaft 10 (the X direction).

The length (depth) y1 [mm] of the sensor element 110 in the Y direction satisfies, for example, 0.05≤y1≤3. The distance y2 [mm] between first portions 701a adjacent in the Y direction (the depth of the opening 702a or the opening 702b) satisfies, for example, 1≤y2≤20. The depth y3 [mm] of the first portion 701a of the sheet 701 satisfies, for example, 0.05≤y1≤3. Although FIG. 23 illustrates an example in which y1 is smaller than y3, y1 may be equal to y3.

The values of the depths y1 to y3 mentioned above are representative ones and hence may vary within the range of the representative value +Δy due to expansion, contraction, or the like. Here, Δy is a value, for example, larger than 0% of the representative value and smaller than 100% of the representative value. For example, Δy is 10% of the representative value.

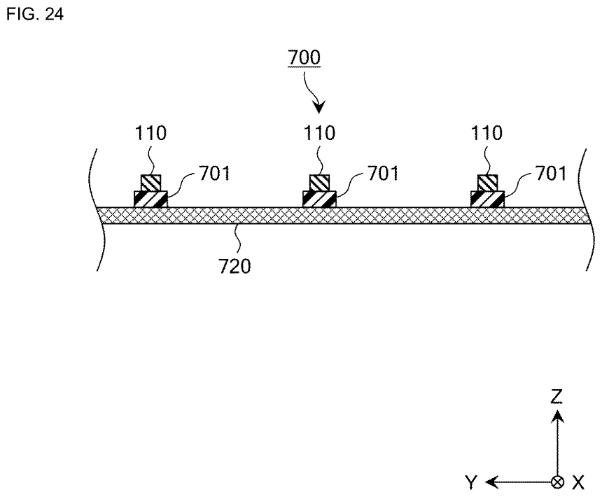

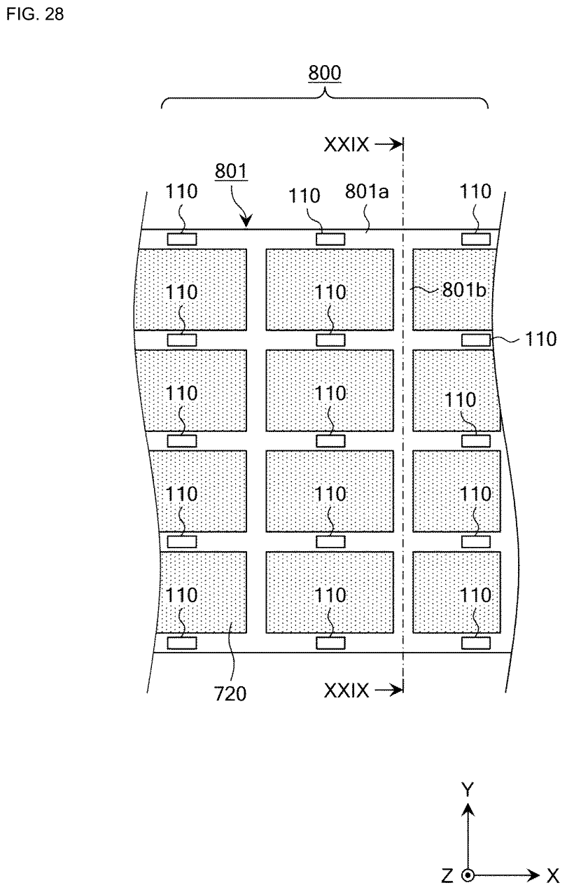

In the present embodiment, the first direction in which the sheet 701 extends is set not to be parallel or orthogonal to the third direction (the Y direction). Thus, when the temperature sensor unit 700 is rolled or folded around the X-axis as a rolling axis to put the temperature sensor unit 700 into the stored state, it is possible to prevent the occurrence of swelling of the roll or reduce the degree of swelling of the roll. The following describes this effect with reference to FIGS. 24 to 27 illustrating an example of the present embodiment and FIGS. 28 to 30 illustrating a comparative example.

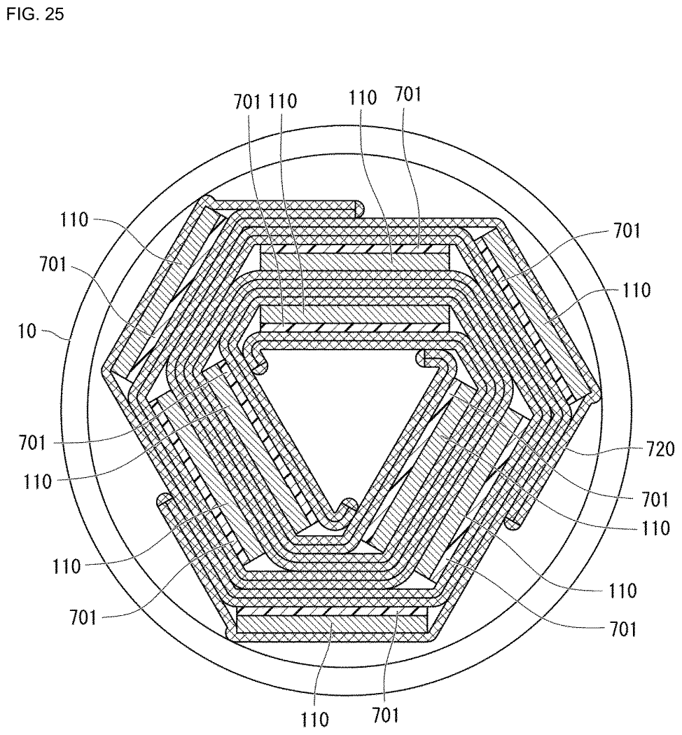

FIG. 24 is a cross-sectional view of the temperature sensor unit 700 and the balloon 720 taken along line XXIV-XXIV in FIG. 23. FIG. 25 is a schematic diagram illustrating the cross section corresponding to FIG. 24 of the temperature sensor unit 700 and the balloon 720 in the stored state.

In the case in which y1=1, y2=5, and y3=1 in FIG. 23, the sensor elements 110 and the sheet 701 are constructed not to be radially aligned in the stored state as illustrated in FIG. 25. This makes it possible to reduce the radial dimension of the temperature sensor unit 700 and the balloon 720 in the stored state.

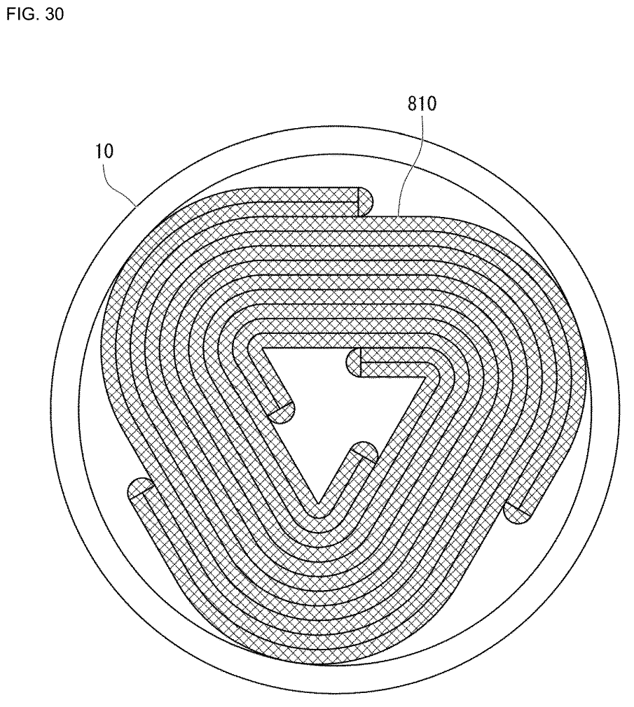

For example, in the case in which the length (thickness) of the sensor element 110 in the Z direction is 80 μm, the thickness of the sheet 701 is 40 μm, and the thickness of the balloon 720 is 50 μm, the temperature sensor unit 700 and the balloon 720 can be stored in a shaft 10 with an inner diameter of 2.706 mm. This is smaller than the dimension of the temperature sensor unit and the balloon of the comparative example described later in the stored state (see FIG. 30). Note that the shaft 10 can be deformed by external pressure. The inner diameter of the shaft 10 mentioned above is the diameter of a circular cross section of the shaft 10 without deformation.

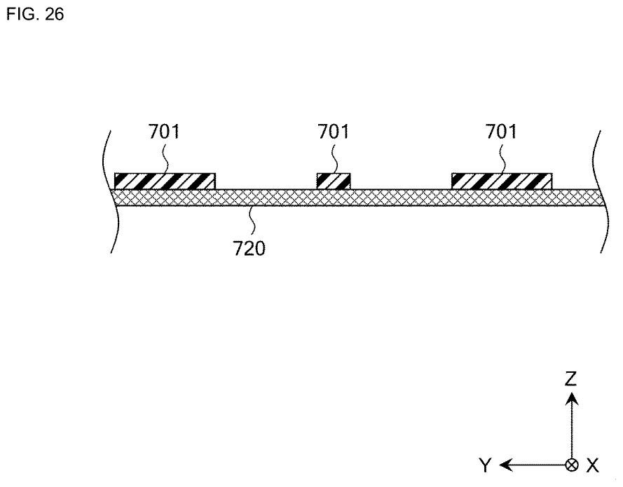

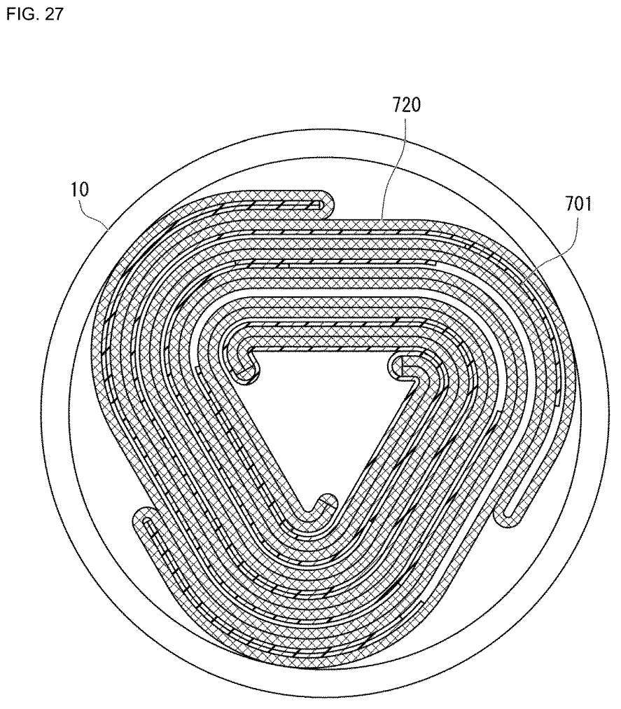

FIG. 26 is a cross-sectional view of the temperature sensor unit 700 and the balloon 720 taken along line XXVI-XXVI in FIG. 23. FIG. 27 is a schematic diagram illustrating the cross section corresponding to FIG. 26 of the temperature sensor unit 700 and the balloon 720 in the stored state.

As illustrated in FIGS. 26 and 27, in the cross section taken along line XXVI-XXVI in FIG. 23, the sheet 701 is not present on some portions of the balloon 720. This configuration prevents swelling of the roll of the temperature sensor unit 700 or reduces the degree of swelling of the roll.

For example, in the case in which y1=1, y2=5, and y3=1 in FIG. 23, and in which the thickness of the sheet 701 is 40 μm, and the thickness of the balloon 720 is 50 μm, the temperature sensor unit 700 and the balloon 720 can be stored in a shaft 10 with an inner diameter of 2.652 mm. This is smaller than the dimension of the temperature sensor unit and the balloon of the comparative example described later in the stored state (see FIG. 30).

FIG. 28 is a schematic diagram illustrating a configuration example of a temperature sensor unit 800 and a balloon 720 according to a comparative example of the present embodiment. The temperature sensor unit 800, as with the temperature sensor unit 700 in FIG. 23, includes a sheet 801 and a plurality of temperature sensor elements 110 located on the sheet 801. The sheet 801 includes a plurality of first portions 801a extending in the X direction and a plurality of second portions 801b extending in the Y direction and intersecting the first portions 801a.

The second portions 801b of the sheet 801, unlike the second portions 701b of the sheet 701 illustrated in FIG. 23, extend parallel to the Y direction.

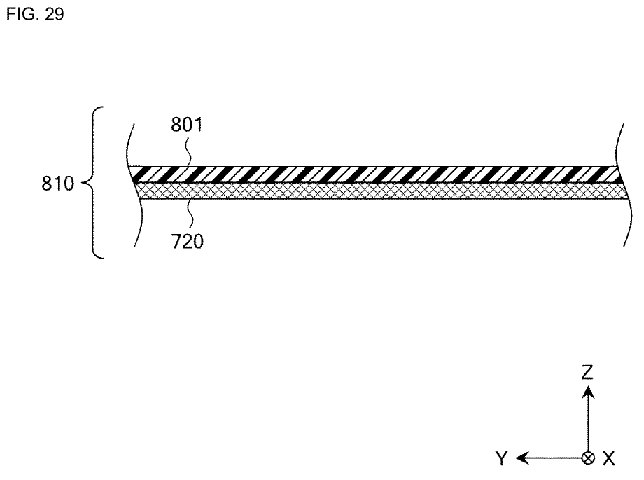

FIG. 29 is a cross-sectional view of the temperature sensor unit 800 and the balloon 720 taken along line XXIX-XXIX in FIG. 28. FIG. 30 is a schematic diagram illustrating the cross section corresponding to FIG. 29 of the temperature sensor unit 800 and the balloon 720 in the stored state.

As compared with the sheet 701 in FIGS. 26 and 27, the sheet 801 in the comparative example illustrated in FIGS. 29 and 30 is located over the entire surface of the balloon 720 in the cross section taken along line XXIX-XXIX in FIG. 28. In this configuration, when the temperature sensor unit 800 is rolled or folded around the X-axis as a rolling axis to put the temperature sensor unit 800 into the stored state, the temperature sensor unit 800 causes a larger swelling of the roll than the temperature sensor unit 700 according to the present embodiment.

For example, in the case in which y1=1, y2=5, and y3=1, and in which the thickness of the sheet 801 is 40 μm, and the thickness of the balloon 720 is 50 μm, the shaft 10 cannot store the temperature sensor unit 800 and the balloon 720 unless the inner diameter of the shaft 10 is larger than or equal to approximately 3.490 μm.

As described above, in the case in which the sheet 801 continuously extend in the folding direction, the swelling of the roll in the stored state is large, so that the temperature sensor elements 110 cannot be arranged at high density in the shaft 10.

In contrast, in the temperature sensor unit 700 according to the present embodiment, the first direction in which the sheet 701 extends is set not to be parallel or orthogonal to the third direction (the Y direction). Thus, when the temperature sensor unit 700 is rolled or folded around the X-axis as a rolling axis to put the temperature sensor unit 700 into the stored state, it is possible to prevent swelling of the roll or reduce the degree of swelling of the roll. Hence, it is possible to arrange the temperature sensor elements 110 at high density in the shaft 10.

The temperature sensor unit 700 according to the present embodiment is folded in the third direction (the Y direction) such that the first direction in which the sheet 701 extends is not parallel or orthogonal to the third direction (the Y direction). Thus, the present embodiment discloses a method of folding a temperature measurement device in which the temperature sensor unit 700 is folded in the third direction (the Y direction) such that the first direction in which the sheet 701 extends is not parallel or orthogonal to the third direction (the Y direction).

Although the present embodiment illustrates an example of the balloon 720 and the temperature sensor unit 700 constructed to be folded, the present embodiment is not limited to this example. For example, a temperature sensor unit according to another example of the present embodiment may be rolled on a balloon to be stored in the shaft 10 as illustrated in FIG. 7. Also in the case in which the temperature sensor unit is stored in this manner, the configuration in which the first direction in which the sheet 701 extends is not parallel or orthogonal to the third direction (the Y direction) reduces the thickness of layers of the sheet 701 when rolled around the X-axis as a rolling axis. Thus, it is possible to prevent the occurrence of swelling of the roll or reduce the degree of swelling of the roll, which makes it possible to arrange the temperature sensor elements 110 at high density in the shaft 10.

### Modification Example

Although the embodiments of the present disclosure have been described above in detail, the description above is mere examples of the present disclosure in every respect. Hence, various improvements and modifications can be made without departing from the scope of the present disclosure. For example, changes as described below can be made. Note that in the following, constituents the same as or similar to the ones in the aforementioned embodiments are denoted by the same or similar symbols, and in points the same as or similar to the ones in the aforementioned embodiments, description thereof is omitted as appropriate. The following modification examples may be combined as appropriate.

### First Modification Example

Although in the description of the aforementioned embodiments, an example of a tubular organ into which the shaft 10 is inserted is the esophagus, the present disclosure is not limited to this example. For example, a tubular organ may be a lumen, a hollow organ, or the like in a living body. Specifically, a tubular organ into which the shaft 10 is inserted may be the trachea, lung, oral cavity, stomach, intestine, external auditory canal, eustachian tube, blood vessel, urinary tract, lymphatic tube, or the like. The tubular organ is not limited to human organs and may be organs of another living thing.

### Second Modification Example

In the first embodiment, a description was given of the sheet 101 having the cuts 103 to be deployed (see FIG. 4). However, the sheet 101 is not limited to this configuration and may be any sheet that can be radially deployed. For example, the sheet 101 may be a stent. Alternatively, the sheet 101 may be a sheet having a structure the same as or similar to that of a stent. In addition, the sheet 101 may have slits having widths in the stored state instead of cuts having no width illustrated as an example in FIG. 4.

### Third Modification Example

Although the second embodiment has been described based on an example in which the surface of the sheet of the temperature sensor unit 200 is hydrophilic, the present disclosure is not limited to this example. For example, the surface of the sheet of the temperature sensor unit may be water-repellent. For example, the surface of the sheet is formed of a water-repellent material to be water-repellent. Alternatively, the surface of the sheet may be water-repellent by being processed to be water-repellent.

In this specification, “water-repellent” denotes such a property of a target surface that when the contact angle θ between water and the target surface is measured according to the aforementioned method indicated in (1) to (3), 90 degrees<θ<180 degrees.

In the case in which the surface of the sheet of the temperature sensor unit is water-repellent, even in the case in which the temperature sensor unit is stored at high density in a catheter in the stored state, when the temperature sensor unit is pushed outward by an expansion member, portions of the sheet in contact with one another can be easily apart.

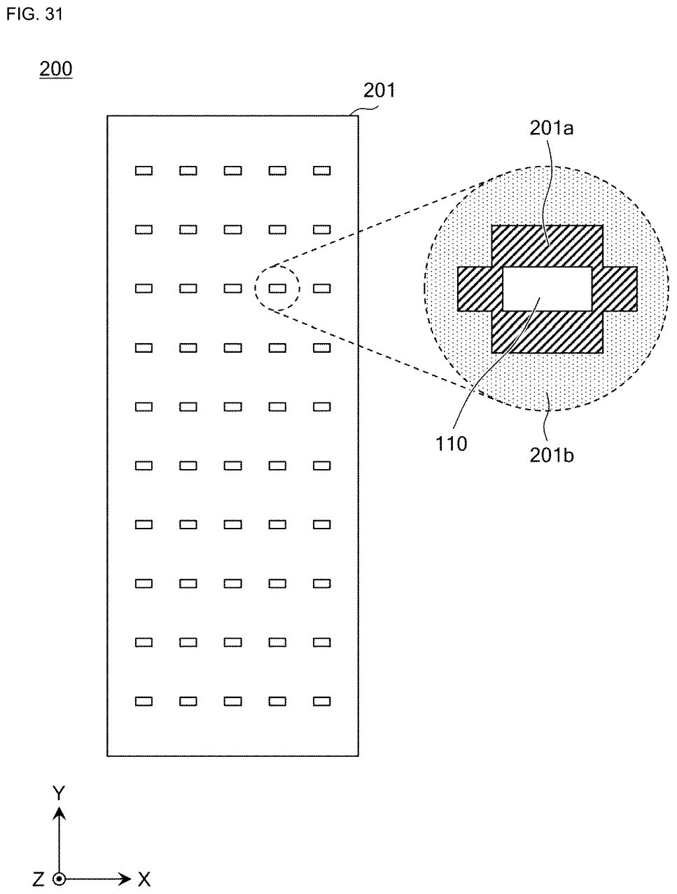

Alternatively, the surface of the sheet of the temperature sensor unit may include hydrophilic portions that are hydrophilic and water-repellent portions that are water-repellent. FIG. 31 is a schematic diagram illustrating a configuration example of a temperature sensor unit 200 according to a modification example of such an embodiment. FIG. 31 also shows a partially enlarged view of the region surrounded by a dashed line. A sheet 201 of a temperature sensor unit 200 includes hydrophilic portions 201a and water-repellent portions 201b. A hydrophilic portion 201a is located around each temperature sensor element 110 in plan view. A water-repellent portion 201b is located around each hydrophilic portion 201a in plan view.

The deployment unit such as the balloon mentioned in the above embodiments may be contracted after being deployed. In the case in which the deployment unit is contracted, it is possible to avoid the esophagus being expanded in the width directions. Contracting the deployment unit makes it possible to avoid the deployment unit pressing the inner wall of the esophagus against the heart, in particular, the left atrial. With this configuration, it is possible to prevent heat from being excessively transmitted from the heart to the esophagus while ablation is performed on the heart.

### Fourth Modification Example

The above embodiments illustrate examples of a temperature measurement device that is inserted into the esophagus to monitor the temperature inside the esophagus during left atrial ablation. However, the applications of the temperature measurement device according to the present disclosure are not limited to these examples. The temperature measurement device according to the present disclosure is applicable to treatment devices used for treatment such as left atrial ablation. In addition, the temperature measurement device according to the present disclosure is applicable to both a treatment device and a device to monitor the temperature inside the esophagus, which are used when a treatment such as left atrial ablation is performed.

Examples of such a treatment device include a cryoballoon which is inserted into the left atrial and used for cryoablation on a myocardial tissue such as the pulmonary vein and a hot balloon for cauterizing a myocardial tissue. In a treatment using a cryoballoon, for example, the balloon is cooled to −60° C. or so with a cooling gas, and myocardial tissue around the balloon is frozen and necrotized. In a treatment using a hot balloon, for example, a high frequency is applied to electrodes in the balloon to heat the liquid injected in the balloon, and myocardial tissue around the balloon is cauterized.

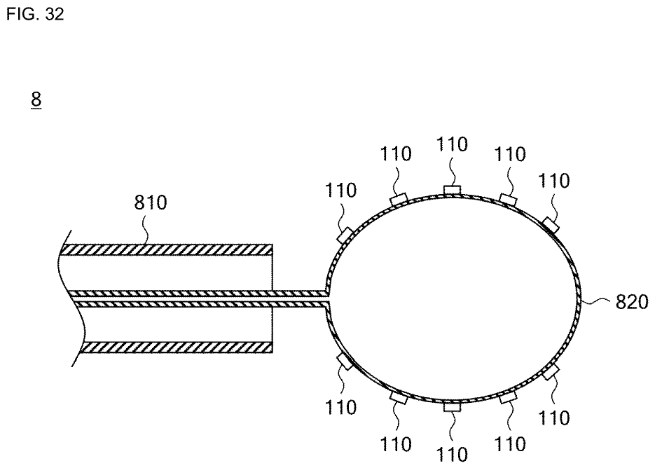

FIG. 32 is a schematic cross-sectional view of a configuration example of a temperature measurement device 8 according to the present modification example. The temperature measurement device 8 is included in a treatment device. A balloon 820 stored in a shaft 810 is transported into the left atrial, then comes out of the shaft 810, and is put into an expanded state illustrated in FIG. 32. The inside of the balloon 820 is filled with a gas or liquid. In the case in which the balloon 820 is used as a cryoballoon, the gas in the balloon 820 is cooled to −60° C. or so. In the case in which the balloon 820 is used as a hot balloon, the liquid in the balloon 820 is heated to 60 to 70° C. or so.

In conventional cryoballoons and hot balloons, temperature sensors are not located on a balloon portion that comes into contact with a myocardial tissue such as the pulmonary vein and are located on a shaft in the balloon. In contrast, in the present modification example, a plurality of temperature sensor elements 110 are located on the outer surface of the balloon 820. This configuration enables the temperature sensor elements 110 to come into contact with a tissue such as the inner surface of the pulmonary vein during treatment and to directly measure the temperature of the tissue. Thus, the temperature measurement device 8 can measure the temperature of a tissue with higher accuracy than in the case in which the temperature of the tissue is indirectly measured by measuring the temperature of the gas or liquid in the balloon 820.

An insulation material may be provided between the gas or liquid in the balloon 820 and the temperature sensor elements 110. For example, in the case in which an insulation material is located on the inner surface and/or the outer surface of the balloon 820, the insulation material prevents the temperature of the gas or liquid in the balloon 820 from being transmitted to the temperature sensor elements 110. This configuration reduces the degree of the influence of the temperature of the gas or liquid in the balloon 820 exerted on the measurement of the temperature of a myocardial tissue such as the pulmonary vein by each temperature sensor element 110. Hence, it is possible to measure the temperature of the tissue with higher accuracy.

### Aspects of Present Disclosure

Hereinafter, aspects of the present disclosure are appended.

<1> A temperature measurement device including: a tube; and a temperature sensor unit within the tube, wherein the temperature sensor unit includes a flexible sheet and a plurality of temperature sensor elements on the flexible sheet.

<2> The temperature measurement device according to <1>, in which the temperature sensor unit is constructed to transition between a stored state in which the temperature sensor unit is stored in the tube and a deployed state in which the temperature sensor unit is deployed outside the tube.

<3> The temperature measurement device according to <2>, in which the tube has a distal end and a proximal end, the temperature measurement device further includes a guide member that moves the temperature sensor unit from an inside of the tube to an outside of the tube through the distal end.

<4> The temperature measurement device according to <1>, further including an expansion member constructed to be stored in the tube, in which the expansion member is constructed to radially outwardly so as to expand the temperature sensor unit.

<5> The temperature measurement device according to <4>, in which the temperature sensor unit is located around at least part of the expansion member.

<6> The temperature measurement device according to <4>, in which at least part of the expansion member is located around the temperature sensor unit.

<7> The temperature measurement device according to <6>, in which the flexible sheet has a first crease, and the plurality of temperature sensor elements are on the flexible sheet and not located over the first crease.

<8> The temperature measurement device according to any one of <4> to <7>, in which the expansion member is a balloon.

<9> The temperature measurement device according to <8>, in which the balloon has a second crease and is constructed to be stored in the tube when folded along the second crease, and the temperature sensor unit is in contact with the balloon, and the plurality of temperature sensor elements are located not to overlap the second crease.

<10> The temperature measurement device according to any one of <4> to <7>, in which the expansion member includes a plurality of wires each extending in a direction from a first end toward a second end of the tube and constructed to curve and protrude in a direction from an inside toward an outside of the tube in a cross-sectional view in a direction intersecting the direction from the first end toward the second end of the tube.

<11> The temperature measurement device according to any one of <1> to <10>, in which a surface of the temperature sensor unit is hydrophilic.

<12> The temperature measurement device according to any one of <1> to <10>, in which a surface of the temperature sensor unit is water-repellent.

<13> The temperature measurement device according to any one of <1> to <10>, in which a surface of the temperature sensor unit includes hydrophilic portions that are hydrophilic and water-repellent portions that are water-repellent.

<14> The temperature measurement device according to <13>, in which one of the hydrophilic portions is located around each temperature sensor element, and one of the water-repellent portions is located around each hydrophilic portion.

<15> The temperature measurement device according to any one of <1> to <14>, in which the temperature sensor unit includes a thermistor, a thermocouple, a semiconductor temperature sensor, or a temperature sensor element including a combination of some of these.

<16> The temperature measurement device according to any one of <1> to <15>, further including a protective layer covering the temperature sensor unit.

<17> The temperature measurement device according to <16>, in which the protective layer contains a metal.

<18> The temperature measurement device according to any one of <1> to <17>, in which the temperature sensor unit is constructed to transition to a deployed state in which the temperature sensor unit is deployed outside the tube, and the plurality of temperature sensor elements are located such that a distance between adjacent temperature sensor elements of the plurality of temperature sensor elements is smaller than or equal to a specified value in the deployed state.

<19> The temperature measurement device according to any one of <1> to <18>, in which the temperature sensor unit includes four or more temperature sensor elements, the temperature sensor unit is constructed to transition to a deployed state in which the temperature sensor unit is deployed outside the tube, the four or more temperature sensor elements in the deployed state compose row sensor-element sets and column sensor-element sets each including two or more of the temperature sensor elements aligned in a row direction and a column direction, respectively, on the temperature sensor unit, the row direction and the column direction intersecting each other, the two or more of the temperature sensor elements in each row sensor-element set in the deployed state are aligned at intervals of a first distance in a direction from a first end toward a second end of the tube, the two or more of the temperature sensor elements in each column sensor-element set in the deployed state are aligned at intervals of a second distance in a direction intersecting a direction from an inside toward an outside of the tube in cross-sectional view in a direction intersecting the direction from the first end toward the second end of the tube, and the first distance is shorter than the second distance.

<20> The temperature measurement device according to any one of <1> to <19>, further including an expansion member constructed to be stored in the tube and constructed to expand in a direction intersecting an extending direction of the tube, which is a direction from a first end toward a second end of the tube, in which the flexible sheet is located on the expansion member, at least part of the flexible sheet is line-shaped and extends in a first direction, and the first direction is not parallel to the extending direction of the tube and is not orthogonal to the extending direction of the tube.

<21> The temperature measurement device according to <20>, in which the flexible sheet includes a plurality of line-shaped first portions extending approximately parallel to the extending direction of the tube and a line-shaped second portion extending in the first direction and connecting the plurality of first portions.

<22> The temperature measurement device according to <20> or <21>, in which the flexible sheet further includes a line-shaped portion extending in a second direction different from the first direction, and the second direction is not parallel to the extending direction of the tube and not orthogonal to the extending direction of the tube.

<23> A treatment device including the temperature measurement device according to any one of <1> to <22>.

### REFERENCE SIGNS LIST

1 TO 6, 8 TEMPERATURE MEASUREMENT DEVICE
10, 810 SHAFT
11 DISTAL END
12 PROXIMAL END
20 BALLOON
30 GUIDE MEMBER
31 GAS FLOW PATH
100, 200, 300, 301, 400, 500, 600, 700 TEMPERATURE SENSOR UNIT
101, 201, 401, 501, 701 SHEET
102 ARM PORTION
105 PROTECTIVE LAYER
110 TEMPERATURE SENSOR ELEMENT
110a ROW SENSOR-ELEMENT SET
110b COLUMN SENSOR-ELEMENT SET
201a HYDROPHILIC PORTION
201b WATER-REPELLENT PORTION
520 BALLOON
520a FIRST PORTION
520b SECOND PORTION
520c THIRD PORTION
521, 522 CREASE
602, 603 CREASE
620 BASKET CATHETER
621 WIRE
622 BINDING PORTION
623 BASKET PORTION
624 SPACE
630 GUIDE MEMBER
720 BALLOON

1. A temperature measurement device comprising:
a tube; and
a temperature sensor unit within the tube, wherein the temperature sensor unit includes a flexible sheet and a plurality of temperature sensor elements on the flexible sheet.

2. The temperature measurement device according to claim 1, wherein the temperature sensor unit is constructed to transition between a stored state in which the temperature sensor unit is stored in the tube and a deployed state in which the temperature sensor unit is deployed outside the tube.

3. The temperature measurement device according to claim 2, wherein
the tube has a distal end and a proximal end,
the temperature measurement device further includes a guide member that moves the temperature sensor unit from an inside of the tube to an outside of the tube through the distal end.

4. The temperature measurement device according to claim 1, further comprising
an expansion member in the tube, wherein
the expansion member is constructed to expand radially outwardly so as to expand the temperature sensor unit.

5. The temperature measurement device according to claim 4, wherein the temperature sensor unit is located around at least part of the expansion member.

6. The temperature measurement device according to claim 4, wherein at least part of the expansion member is located around the temperature sensor unit.

7. The temperature measurement device according to claim 6, wherein
the flexible sheet has a first crease, and
the plurality of temperature sensor elements are on the flexible sheet and not located over the first crease.

8. The temperature measurement device according to claim 4, wherein the expansion member is a balloon.

9. The temperature measurement device according to claim 8, wherein
the balloon has a second crease and is constructed to be stored in the tube when folded along the second crease, and
the temperature sensor unit is in contact with the balloon, and the plurality of temperature sensor elements are located not to overlap the second crease.

10. The temperature measurement device according to claim 4, wherein the expansion member includes a plurality of wires each extending in a direction from a first end toward a second end of the tube and constructed to curve and protrude in a direction from an inside toward an outside of the tube in a cross-sectional view in a direction intersecting the direction from the first end toward the second end of the tube.

11. The temperature measurement device according to claim 1, wherein a surface of the temperature sensor unit includes hydrophilic portions that are hydrophilic and water-repellent portions that are water-repellent.

12. The temperature measurement device according to claim 1, wherein the temperature sensor unit includes a thermistor, a thermocouple, a semiconductor temperature sensor, a temperature sensor element, or a combination thereof.

13. The temperature measurement device according to claim 1, further comprising a protective layer covering the temperature sensor unit.

14. The temperature measurement device according to claim 13, wherein the protective layer contains a metal.

15. The temperature measurement device according to claim 1, wherein
the temperature sensor unit is constructed to transition to a deployed state in which the temperature sensor unit is deployed outside the tube, and
the plurality of temperature sensor elements are located such that a distance between adjacent temperature sensor elements of the plurality of temperature sensor elements is smaller than or equal to a specified value in the deployed state.

16. The temperature measurement device according to claim 1, wherein
the temperature sensor unit includes four or more temperature sensor elements,
the temperature sensor unit is constructed to transition to a deployed state in which the temperature sensor unit is deployed outside the tube,
the four or more temperature sensor elements in the deployed state compose row sensor-element sets and column sensor-element sets each including two or more of the temperature sensor elements aligned in a row direction and a column direction, respectively, on the temperature sensor unit, the row direction and the column direction intersecting each other,
the two or more of the temperature sensor elements in each row sensor-element set in the deployed state are aligned at intervals of a first distance in a direction from a first end toward a second end of the tube,
the two or more of the temperature sensor elements in each column sensor-element set in the deployed state are aligned at intervals of a second distance in a direction intersecting a direction from an inside toward an outside of the tube in a cross-sectional view in a direction intersecting the direction from the first end toward the second end of the tube, and
the first distance is shorter than the second distance.

17. The temperature measurement device according to claim 1, further comprising
an expansion member constructed to be stored in the tube and constructed to expand in a direction intersecting an extending direction of the tube, which is a direction from a first end toward a second end of the tube, wherein
the flexible sheet is located on the expansion member,
at least part of the flexible sheet is line-shaped and extends in a first direction, and
the first direction is not parallel to the extending direction of the tube and is not orthogonal to the extending direction of the tube.

18. The temperature measurement device according to claim 17, wherein the flexible sheet includes a plurality of line-shaped first portions extending approximately parallel to the extending direction of the tube and a line-shaped second portion extending in the first direction and connecting the plurality of first portions.

19. The temperature measurement device according to claim 17, wherein
the flexible sheet further includes a line-shaped portion extending in a second direction different from the first direction, and
the second direction is not parallel to the extending direction of the tube and not orthogonal to the extending direction of the tube.

20. A treatment device comprising the temperature measurement device according to claim 1.
